(19) United States
(12) Patent Application Publication
(10) Pub. No.: US 2024/0166676 A1
(43) Pub. Date: May 23, 2024

(54) GROUP 6 AMIDINATE PADDLEWHEEL COMPOUNDS FOR DEPOSITION OF METAL CONTAINING THIN FILMS

(71) Applicants: Merck Patent GmbH, Darmstadt (DE); Versum Materials US, LLC, Tempe, AZ (US)

(72) Inventors: Jason P. COYLE, San Jose, CA (US); Michael T. SAVO, Bethlehem, PA (US); Sergei V. IVANOV, Schnecksville, PA (US); Alan C. COOPER, Macungie, PA (US)

(21) Appl. No.: 18/263,011

(22) PCT Filed: Feb. 11, 2022
(86) PCT No.: PCT/US2022/016044

§ 371 Date: Jul. 26, 2023

Related U.S. Application Data
(60) Provisional application No. 63/149,524, filed on Feb. 15, 2021.

(51) Int. Cl. C07F 11/00; C23C 16/18; C23C 16/36; C23C 16/455; C23C 16/50

(52) U.S. Cl. CPC C07F 11/005; C23C 16/18; C23C 16/36; C23C 16/45553; C23C 16/50

(57) ABSTRACT

The disclosed and claimed subject matter relates to organometallic amidinate and guanidinate paddlewheel compounds, compositions containing the compounds and methods of using the compounds as precursors for deposition of metal-containing films.

### BACKGROUND

### Field

The disclosed and claimed subject matter relates to organometallic amidinate and guanidinate paddlewheel compounds, compositions containing the compounds and methods of using the compounds as precursors for deposition of metal-containing films.

### Related Art

Transition metal-containing films are used in semiconductor and electronics applications. Chemical Vapor Deposition (CVD) and Atomic Layer Deposition (ALD) have been applied as the main deposition techniques for producing thin films for semiconductor devices. These methods enable the achievement of conformal films (metal, metal oxide, metal nitride, metal silicide, and the like) through chemical reactions of metal-containing compounds (precursors). The chemical reactions occur on surfaces which may include metals, metal oxides, metal nitrides, metal silicides, and other surfaces. In CVD and ALD, the precursor molecule plays a critical role in achieving high quality films with high conformality and low impurities. The temperature of the substrate in CVD and ALD processes is an important consideration in selecting a precursor molecule. Higher substrate temperatures, in the range of 150 to 500 degrees Celsius (° C.), promote a higher film growth rate. The preferred precursor molecules must be stable in this temperature range. The preferred precursor is capable of being delivered to the reaction vessel in a liquid phase. Liquid phase delivery of precursors generally provides a more uniform delivery of the precursor to the reaction vessel than solid phase precursors.

CVD and ALD processes are increasingly used as they have the advantages of enhanced compositional control, high film uniformity, and effective control of doping. Moreover, CVD and ALD processes provide excellent conformal step coverage on highly non-planar geometries associated with modern microelectronic devices.

CVD is a chemical process whereby precursors are used to form a thin film on a substrate surface. In a typical CVD process, the precursors are passed over the surface of a substrate (e.g., a wafer) in a low pressure or ambient pressure reaction chamber. The precursors react and/or decompose on the substrate surface creating a thin film of deposited material. Plasma can be used to assist in reaction of a precursor or for improvement of material properties. Volatile by-products are removed by gas flow through the reaction chamber. The deposited film thickness can be difficult to control because it depends on coordination of many parameters such as temperature, pressure, gas flow volumes and uniformity, chemical depletion effects, and time.

ALD is a chemical method for the deposition of thin films. It is a self-limiting, sequential, unique film growth technique based on surface reactions that can provide precise thickness control and deposit conformal thin films of materials provided by precursors onto surfaces substrates of varying compositions. In ALD, the precursors are separated during the reaction. The first precursor is passed over the substrate surface producing a monolayer on the substrate surface. Any excess unreacted precursor is pumped out of the reaction chamber. A second precursor or co-reactant is then passed over the substrate surface and reacts with the first precursor, forming a second monolayer of film over the first-formed monolayer of film on the substrate surface. Plasma may be used to assist with reaction of a precursor or co-reactant or for improvement in materials quality. This cycle is repeated to create a film of desired thickness.

Thin films, and in particular thin metal-containing films, have a variety of important applications, such as in nanotechnology and the fabrication of semiconductor devices. Examples of such applications include capacitor electrodes, gate electrodes, adhesive diffusion barriers, and integrated circuits.

For forming molybdenum nitride films, the compound Mo(VI)(NMe₂)₂(NtBu)₂:

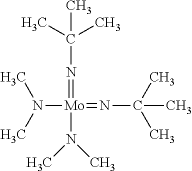

has been used to prepare nitride films. See Chem. Mater., 19, 263-269 (2007). The precursor is commonly referred to as MoBure. MoBure is evaporated below 100° C. and deposits molybdenum nitride films via thermal atomic layer deposition with ammonia. The growth rate was 0.5 Å/cycle within the reactor temperature range of 260-300° C. The film composition contains a Mo:N ratio of 1:1 and was predominately amorphous. Resistivity values of the thin film were not reported. Internal experiments have measured resistivities of >2000 μΩ·cm for MoBure.

Other methods for forming thin films from MoBure of molybdenum carbide and carbonitride films by plasma-enhanced atomic layer deposition and chemical vapor deposition with hydrogen have been reported. The electrical resistivity of such films (deposited at 150° C. by plasma-enhanced process) was between 170-200 μΩ·cm. See J. Vac. Sci. Technol., A35, 01B141 (2017) and Thin Solid Films, 692, 137607 (2017). No literature was identified describing such films, e.g., molybdenum-containing films, with such low resistivity deposited from halogen-free metal-containing precursors are not known. Typically, a plasma-enhanced process is required to achieve low resistivity from halogen-free molybdenum and tungsten precursors.

Molybdenum paddlewheel compounds are generally known in the literature. Although the known compounds have not been studied as precursors for ALD and CVD. In fact, most of the examples contain aromatic substituents that undesirably affect the key physical property of precursor volatility. The best-known example of a molybdenum paddlewheel compound with potential for ALD and CVD application is Mo₂(OAc){(NiPr)₂CMe}₃ by Yamaguchi, Y. et al., Inorganica Chim. Acta., 358, 2363-2370 (2005). However, as those skilled in the art recognize and understand, the presence of an acetate ligand in such compounds is a source for oxygen impurities that can be detrimental to applications requiring thin films with low resistivity. The disclosed and claimed subject matter, therefore, provides Group 6 (i.e., chromium, molybdenum and tungsten) paddlewheel compounds synthesized without acetate ligands. These new paddlewheel precursors are thermally stable and are suitable as CVD and ALD precursors that can be preferably delivered in liquid phase, have low impurities and can produce a high-quality film with high conformality and low resistivity.

The synthesis of the acetate-free paddlewheel compounds disclosed and claimed herein was determined to be dependent upon correctly choosing the amidinate or guanidinate ligand. Without intending or being bound by theory, when following the synthesis method from literature, complete replacement of all four acetate ligands is dependent upon the steric bulk of the amidinate or guanidinate ligand.

### SUMMARY

The disclosed and claimed subject matter pertains to amidinate and guanidinate paddlewheel compounds of chromium, molybdenum and tungsten for use as ALD and CVD precursors.

In one embodiment, the precursors are amidinate (“Ad”) paddlewheel compounds have the general Formula I shown below

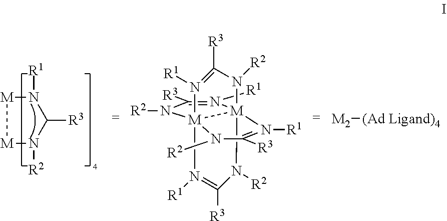

where: M is one of chromium, molybdenum and tungsten; and
   R¹, R² and R³ are each independently selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen and a C₃-C₈ aromatic group substituted with an amino group. In one aspect of this embodiment, all four amidinate ligands have the same chemical structure. In another aspect of this embodiment, two or more of the amidinate ligands have the same chemical structure. In another aspect of this embodiment, all four amidinate ligands have a different chemical structure.

In additional aspects of this embodiment, the compounds of Formula I include heterocyclic Ad ligands (Formula II-A and Formula II-B) and/or heterocyclic bicyclic Ad ligands (Formula II-C) as shown below where one or both of (a) R¹ and R³ and (b) R² and R³ independently constitute parts of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen.

In one aspect, the precursor has Formula II-A:

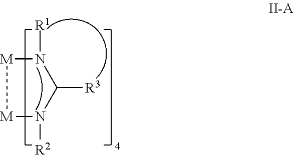

where: M is one of chromium, molybdenum and tungsten;
   R² is selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen, a C₃-C₈ aromatic group substituted with an amino group; and
   R¹ and R³ constitute part of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen.

In one aspect, the precursor has Formula II-B:

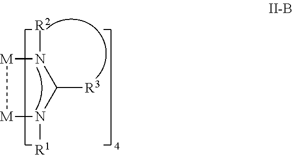

where: M is one of chromium, molybdenum and tungsten;
   R¹ is selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen, a C₃-C₈ aromatic group substituted with an amino group; and
   R² and R³ constitute part of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen.

In one aspect, the precursor has Formula II-B:

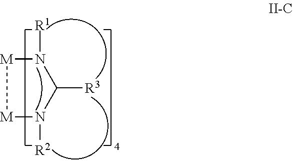

where: M is one of chromium, molybdenum and tungsten; and
   each of (a) R¹ and R³ and (b) R² and R³ independently constitute parts of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen.

As those this skilled in the art will understand, the backbone of the alkylenes and heteroalkylenes in each 5- or 6-membered ring described in Formula II-A and Formula II-B and/or Formula II-C above will contain either three or four atoms exclusive of any substituents or pendent chains thereon.

In another embodiment the precursors are guanidinate (“Gd”) paddlewheel compounds have the general Formula III shown below

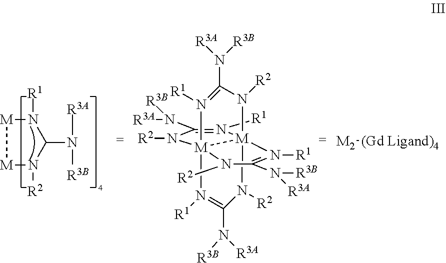

where: M is one of chromium, molybdenum and tungsten; and
   R¹, R², R^{3A} and R^{3B} are each independently selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen and a C₃-C₈ aromatic group substituted with an amino group. In one aspect of this embodiment, all four guanidinate ligands have the same chemical structure. In another aspect of this embodiment, two or more of the guanidinate ligands have the same chemical structure. In another aspect of this embodiment, all four guanidinate ligands have a different chemical structure.

In additional aspects of this embodiment, the compounds of Formula III include heterocyclic Gd ligands (Formula IV-A and Formula IV-B) and/or heterocyclic bicyclic Gd ligands (Formula IV-C) as shown below where one or both of (a) R¹ and R³ and (b) R² and R³ independently constitute parts of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen.

In one aspect, the precursor has Formula IV-A:

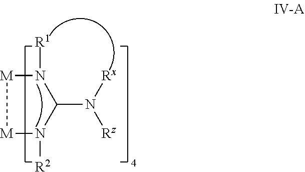

where: M is one of chromium, molybdenum and tungsten;
   R² is selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen, a C₃-C₈ aromatic group substituted with an amino group; and
   R¹ R¹ and R^{X} constitute part of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen, where R^{Z} is one of R^{3A} and R^{3B} and R^{X} is the other of R^{3A} and R^{3B} that is not connected by a linking group to R¹.

In one aspect, the precursor has Formula IV-B:

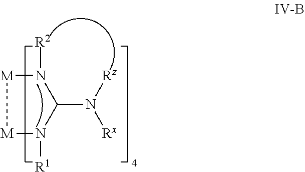

where: M is one of chromium, molybdenum and tungsten;
   R¹ is selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen, a C₃-C₈ aromatic group substituted with an amino group; and
   R² and R^{Z} constitute part of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen, where R^{Z} is one of R^{3A} and R^{3B} and R^{X} is the other of R^{3A} and R^{3B} that is not connected by a linking group to R².

In one aspect, the precursor has Formula IV-C:

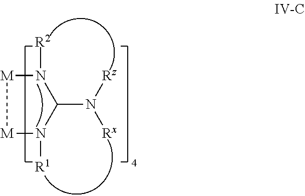

where: M is one of chromium, molybdenum and tungsten; and
   each of (a) R¹ and R^{X} and (b) R² and R^{Z} independently constitute parts of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen, where IV is one of R^{3A} and R^{3B} and R^{X} is the other of R^{3A} and R^{3B}.

As those this skilled in the art will understand, the backbone of the alkylenes and heteroalkylenes in each 5- or 6-membered ring described in Formula IV-A and Formula IV-B and/or Formula IV-C above will contain either three or four atoms exclusive of any substituents or pendent chains thereon.

The above precursors, and the more specifically described embodiments below, of compound provide halide-free and oxygen-free precursors for applications where such contaminates are detrimental.

The disclosed and claimed subject further includes (i) compositions and formulations that include the disclosed and claimed precursors, (ii) methods of using the disclosed and claimed precursors in deposition processes and (iii) metal-containing films derived from the disclosed and claimed precursors produced in deposition processes. The method produces thin films with improved properties compared to known methods which could be attributed to the low oxidation state of the paddlewheel precursors. Such metal and metal-containing thin films can be produced by thermal or plasma ALD and CVD using the disclosed and claimed precursors.

The precursor can be used to produce metal-containing (e.g., molybdenum) thin films under mild conditions. For example, molybdenum carbonitride thin films with low electrical resistivity have been deposited in the absence of a plasma in a thermal ALD process from molybdenum (II) amidinate precursor of the disclosed and claimed subject matter. Traditionally, molybdenum carbonitride thin films produced from MoBure required a plasma-enhanced process. Without intending or being bound by theory, it appears that the “rigid structure” of the paddlewheel compounds very effectively stabilizes the low valent metal atom to afford a compound with thermal stability and volatility. In contrast, for example, MoBure has an (VI) oxidation state which requires a strongly reducing hydrogen plasma in order to deposit thin films with low resistivity.

### BRIEF DESCRIPTION OF THE DRAWINGS

The accompanying drawings, which are included to provide a further understanding of the disclosed subject matter and are incorporated in and constitute a part of this specification, illustrate embodiments of the disclosed subject matter and together with the description serve to explain the principles of the disclosed subject matter. In the drawings:

FIG. 1 illustrates the thermo gravimetric analysis of examples 1 to 4 (where A=Mo₂ (3A)₄; B=Mo₂-(3B)₄; C=Mo₂-(3TT)₄ as illustrated in Table 5 and comparative example Mo₂-(3J)₃(Ac));

FIG. 2 illustrates the proton (¹H) NMR of tetrakis(N,N′-dimethylformamidinate) dimolybdenum of Example 1;

FIG. 3 illustrates the proton (¹H) NMR of tetrakis(N,N′-diethylformamidinate) dimolybdenum of Example 2;

FIG. 4 illustrates the proton (¹H) NMR of tetrakis(N-sec-butyliminopyrrolydinate) dimolybdenum of Example 3;

FIG. 5 illustrates top down and cross-section SEM of Mo-containing film deposited at 350° C. wafer temperature by NH₃ CCVD process of Example 5: 10 sec of Mo₂ (Et-FMD)₄/30 sec Ar purge/5 sec of NH₃/30 sec of Ar purge;

FIG. 6 illustrates top down and cross-section SEM of Mo-containing film deposited at 350° C. wafer temperature by NH₃ CCVD process of Example 5: 20 sec of Mo₂(Et-FMD)₄/30 sec Ar purge/5 sec of NH₃/30 sec of Ar purge; and

FIG. 7 illustrates Auger depth profile of Mo-containing film deposited at 350° C. wafer temperature by NH₃ CCVD process of Example 5: 10 sec of Mo₂(Et-FMD)₄/30 sec Ar purge/5 sec of NH₃/30 sec of Ar purge.

FIG. 8 illustrates the crystal structure of Mo₂(Me-FMD)₄

FIG. 9 illustrates the crystal structure of Mo₂(Et-FMD)₄

FIG. 10 illustrates a cross-section TEM of Mo-containing film deposited at 360° C. and 375° C. wafer temperature on high aspect ratio patterned wafer by NH₃ ALD process of the Example 11: 20 sec of Mo₂(Et-FMD)₄/30 sec Ar purge/5 sec of NH₃/30 sec of Ar purge;

FIG. 11 illustrates a cross-section TEM of thin continuous film deposited at 400° C. wafer temperature on silicon oxide substrate by NH₃ CCVD process of Example 12: 10 sec of Mo₂ (Me-FMD)₄/30 sec Ar purge/5 sec of NH₃/30 sec of Ar purge;

FIG. 12 illustrates a photo of the silicon oxide wafers after deposition of Mo metal film by MoO₂Cl₂/H₂ thermal ALD on the wafers with and without a seed layer deposited by Mo₂ (Me-FMD)₄, as described in the example 15;

FIG. 13 illustrates a cross-section TEM of the low resistivity Mo metal film deposited by MoO₂Cl₂/H₂ thermal ALD on a seed layer deposited by NH₃ CCVD process of Example 16: 10 sec of Mo₂(Me-FMD)₄/30 sec Ar purge/5 sec of NH₃/30 sec of Ar purge;

FIG. 14 illustrates simulated powder X-ray Diffraction (PXRD) spectrum using experimental unit cell parameters of Mo₂(Me-FMD)₄; and

FIG. 15 illustrates simulated powder X-ray Diffraction (PXRD) spectrum using experimental unit cell parameters of Mo₂(Et-FMD)₄.

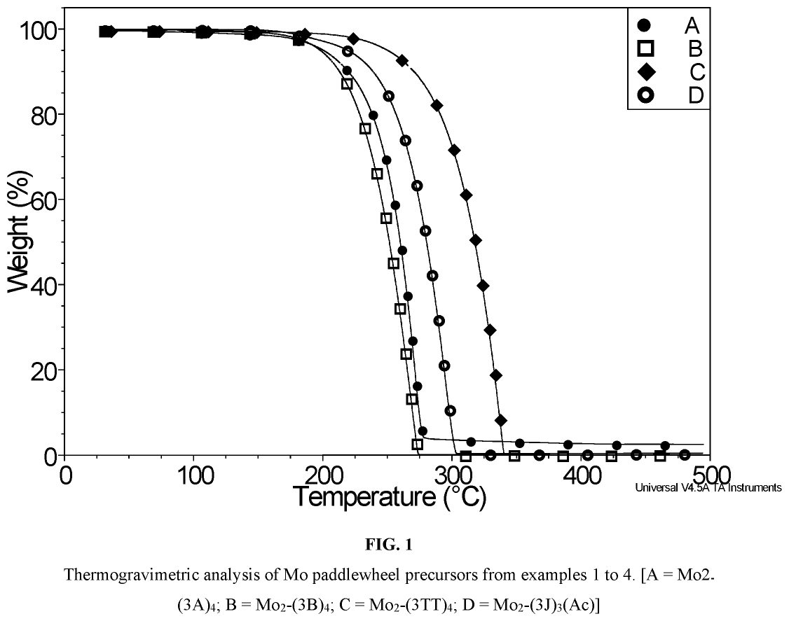

### DETAILED DESCRIPTION

All references, including publications, patent applications, and patents, cited herein are hereby incorporated by reference to the same extent as if each reference were individually and specifically indicated to be incorporated by reference and were set forth in its entirety herein. The use of the terms “a” and “an” and “the” and similar referents in the context of describing the disclosed and claimed subject matter (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosed and claimed subject matter and does not pose a limitation on the scope of the disclosed and claimed subject matter unless otherwise claimed. No language in the specification should be construed as indicating any non-claimed element as essential to the practice of the disclosed and claimed subject matter. The use of the term “comprising” or “including” in the specification and the claims includes the narrower language of “consisting essentially of” and “consisting of”.

Embodiments of the disclosed and claimed subject matter are described herein, including the best mode known to the inventors for carrying out the disclosed and claimed subject matter. Variations of those embodiments may become apparent to those of ordinary skill in the art upon reading the foregoing description. The inventors expect skilled artisans to employ such variations as appropriate, and the inventors intend for the disclosed and claimed subject matter to be practiced otherwise than as specifically described herein. Accordingly, the disclosed and claimed subject matter includes all modifications and equivalents of the subject matter recited in the claims appended hereto as permitted by applicable law. Moreover, any combination of the above-described elements in all possible variations thereof is encompassed by the disclosed and claimed subject matter unless otherwise indicated herein or otherwise clearly contradicted by context.

It will be understood that the term “silicon” as deposited as a material on a microelectronic device will include polysilicon.

For ease of reference, “microelectronic device” or “semiconductor device” corresponds to semiconductor wafers having integrated circuits, memory, and other electronic structures fabricated thereon, and flat panel displays, phase change memory devices, solar panels and other products including solar substrates, photovoltaics, and microelectromechanical systems (MEMS), manufactured for use in microelectronic, integrated circuit, or computer chip applications. Solar substrates include, but are not limited to, silicon, amorphous silicon, polycrystalline silicon, monocrystalline silicon, CdTe, copper indium selenide, copper indium sulfide, and gallium arsenide on gallium. The solar substrates may be doped or undoped. It is to be understood that the term “microelectronic device” or “semiconductor device” is not meant to be limiting in any way and includes any substrate that will eventually become a microelectronic device or microelectronic assembly.

As defined herein, the term “barrier material” corresponds to any material used in the art to seal the metal lines, e.g., copper interconnects, to minimize the diffusion of said metal, e.g., copper, into the dielectric material. Preferred barrier layer materials include tantalum, titanium, ruthenium, hafnium, and other refractory metals and their nitrides and silicides.

“Substantially free” is defined herein as less than 0.001 wt. %. “Substantially free” also includes 0.000 wt. %. The term “free of” means 0.000 wt. %. As used herein, “about” or “approximately” are intended to correspond to within ±5% of the stated value.

“Ad ligand” means amidinate ligand. “Gd ligand” means guanidinate ligand.

“Alkylene” means a linear saturated divalent hydrocarbon radical of one to six carbon atoms or a branched saturated divalent hydrocarbon radical of three to six carbon atoms unless otherwise stated (e.g., methylene, ethylene, propylene, 1-methylpropylene, 2-methylpropylene, butylene, pentylene, and the like).

“Heteroalkylene” means an -(alkylene)-radical as defined above where one, two or three carbons in the alkylene chain is replaced by —O—, N(H, alkyl, or substituted alkyl), S, SO, SO2, or CO. In some preferred embodiments, the carbons are replaced by O or N.

In all such compositions, wherein specific components of the composition are discussed in reference to weight percentage (or “weight %”) ranges including a zero lower limit, it will be understood that such components may be present or absent in various specific embodiments of the composition, and that in instances where such components are present, they may be present at concentrations as low as 0.001 weight percent, based on the total weight of the composition in which such components are employed. Note all percentages of the components are weight percentages and are based on the total weight of the composition, that is, 100%. Any reference to “one or more” or “at least one” includes “two or more” and “three or more” and so on.

Where applicable, all weight percents unless otherwise indicated are “neat” meaning that they do not include the aqueous solution in which they are present when added to the composition. For example, “neat” refers to the weight % amount of an undiluted acid or other material (i.e., the inclusion 100 g of 85% phosphoric acid constitutes 85 g of the acid and 15 grams of diluent).

Moreover, when referring to the compositions described herein in terms of weight %, it is understood that in no event shall the weight % of all components, including non-essential components, such as impurities, add to more than 100 weight %. In compositions “consisting essentially of” recited components, such components may add up to 100 weight % of the composition or may add up to less than 100 weight %. Where the components add up to less than 100 weight %, such composition may include some small amounts of a non-essential contaminants or impurities. For example, in one such embodiment, the formulation can contain 2% by weight or less of impurities. In another embodiment, the formulation can contain 1% by weight or less than of impurities. In a further embodiment, the formulation can contain 0.05% by weight or less than of impurities. In other such embodiments, the constituents can form at least 90 wt %, more preferably at least 95 wt % , more preferably at least 99 wt %, more preferably at least 99.5 wt %, most preferably at least 99.9 wt %, and can include other ingredients that do not material affect the performance of the wet etchant. Otherwise, if no significant non-essential impurity component is present, it is understood that the composition of all essential constituent components will essentially add up to 100 weight %.

The headings employed herein are not intended to be limiting; rather, they are included for organizational purposes only.

### EXEMPLARY EMBODIMENTS

The disclosed and claimed subject matter pertains to amidinate and guanidinate paddlewheel compounds of chromium, molybdenum and tungsten for use as ALD and CVD precursors.

### Amidinate Paddlewheel Precursors

One aspect of the disclosed and claimed subject matter pertains to amidinate paddlewheel compounds of Formula I:

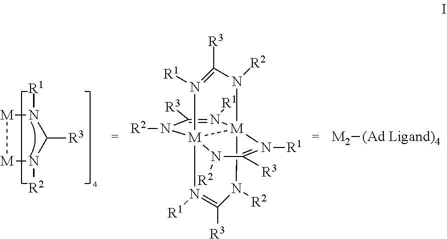

where: M is one of chromium, molybdenum and tungsten; and
   R¹, R² and R³ are each independently selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen and a C₃-C₈ aromatic group substituted with an amino group. In one aspect of this embodiment, all four amidinate ligands have the same chemical structure. In another aspect of this embodiment, two or more of the amidinate ligands have the same chemical structure. In another aspect of this embodiment, all four amidinate ligands have a different chemical structure.

In one aspect of this embodiment, R¹, R² and R³ are each independently selected from H, unsubstituted linear C₁ to C₃ alkyl group and an unsubstituted branched C₃ or C₄ alkyl group. In one aspect, one or more of R¹, R² and R³ is a methyl group. In one aspect, one or more of R¹, R² and R³ is an ethyl group. In one aspect, one or more of R¹, R² and R³ is a propyl group. In one aspect, one or more of R¹, R² and R³ is an isopropyl group. In one aspect, one or more of R¹, R² and R³ is a sec-butyl group. In one aspect, one or more of R¹, R² and R³ is a n-butyl group. In one aspect, one or more of R¹, R² and R³ is an iso-butyl group.

In one aspect of this embodiment, M is chromium. In another aspect of this embodiment, M is molybdenum. In another aspect of this embodiment, M is tungsten.

In some embodiments, the amidinate ligand (“Ad Ligand”) has a structure as illustrated in Table 1:

TABLE 1
Amidinate Ligands

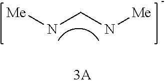

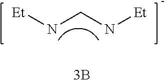

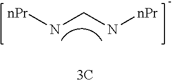

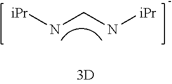

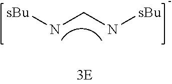

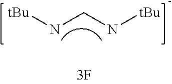

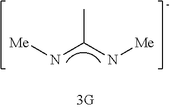

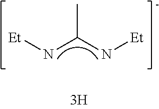

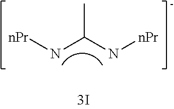

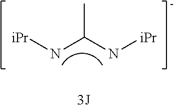

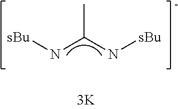

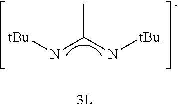

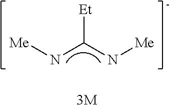

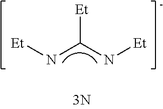

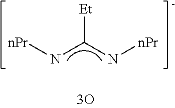

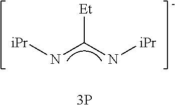

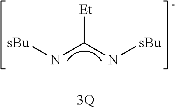

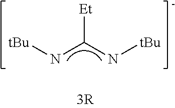

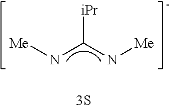

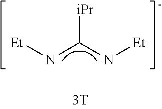

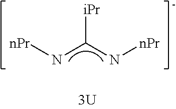

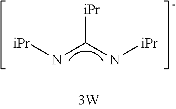

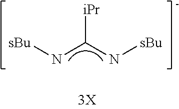

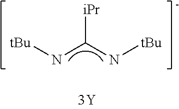

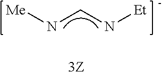

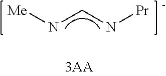

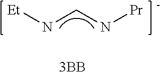

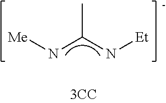

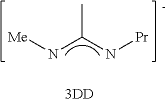

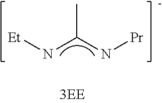

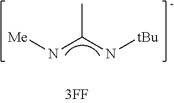

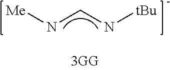

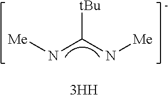

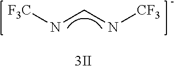

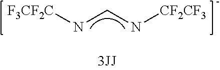

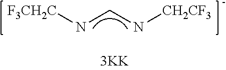

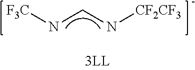

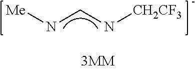

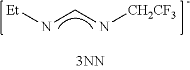

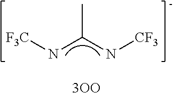

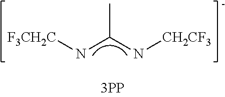

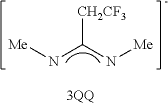

In one aspect of this embodiment, the compounds of Formula I have the following structure where the Ad ligand is a formamidinate ligand:

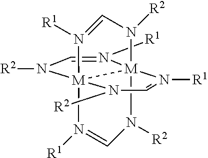

where: M=Cr, Mo, W;
   R¹=a C₁-C₅ substituted or unsubstituted alkyl group; and
   R²=a C₁-C₅ substituted or unsubstituted alkyl group.
      In one example of this embodiment, M=Mo and each of R¹ and R² is a methyl group (—CH₃):

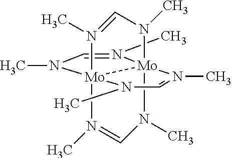

In one example of this embodiment, M=Mo and each of R¹ and R² is an ethyl group (—CH₂CH₃):

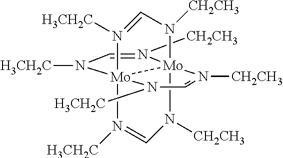

In additional aspects of this embodiment, the compounds of Formula I include heterocyclic Ad ligands (Formula II-A and Formula II-B) and/or heterocyclic bicyclic Ad ligands (Formula II-C) as shown below where one or both of (a) R¹ and R³ and (b) R² and R³ independently constitute parts of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen. In one aspect, the precursor has Formula II-A:

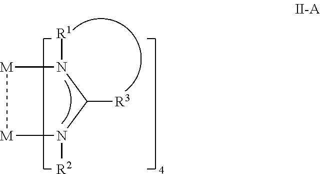

where: M is one of chromium, molybdenum and tungsten;
   R² is selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen, a C₃-C₈ aromatic group substituted with an amino group; and
   R¹ and R³ constitute part of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen.

As those this skilled in the art will understand, the backbone of the alkylenes and heteroalkylenes described in the embodiment above will contain either three or four atoms exclusive of any sub stituents or pendent chains thereon.

In one aspect of this embodiment, R¹ and R³ constitute part of a 5-member heterocyclic ring. In a further aspect, R¹ and R³ are an unsubstituted alkylene linking group containing three carbons. In a further aspect, R¹ and R³ are a substituted alkylene linking group containing three carbons. In a further aspect, R¹ and R³ are a substituted alkylene linking group containing three carbons substituted by at least one halogen atom. In a further aspect, R¹ and R³ are a substituted alkylene linking group containing three carbons substituted by at least one fluorine atom. In a further aspect, R¹ and R³ are a substituted alkylene linking group containing three carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, R¹ and R³ are an unsubstituted heteroalkylene linking group containing two carbons and an oxygen. In a further aspect, R¹ and R³ are an unsubstituted heteroalkylene linking group containing two carbons and a nitrogen. In a further aspect, R¹ and R³ are a substituted heteroalkylene linking group containing two carbons, an oxygen or a nitrogen atom and substituted with a halogen. In a preferred embodiment of this aspect, the halogen is a fluorine. In a further aspect, R¹ and R³ are a substituted heteroalkylene linking group containing two carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups.

In one aspect of this embodiment, R¹ and R³ constitute part of a 6-member heterocyclic ring. In a further aspect, R¹ and R³ are an unsubstituted alkylene linking group containing four carbons. In a further aspect, R¹ and R³ are a substituted alkylene linking group containing four carbons. In a further aspect, R¹ and R³ are a substituted alkylene linking group containing four carbons substituted by at least one halogen atom. In a further aspect, R¹ and R³ are a substituted alkylene linking group containing four carbons substituted by at least one fluorine atom. In a further aspect, R¹ and R³ are a substituted alkylene linking group containing four carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, R¹ and R³ are an unsubstituted heteroalkylene linking group containing three carbons and an oxygen. In a further aspect, R¹ and R³ are an unsubstituted heteroalkylene linking group containing three carbons and a nitrogen. In a further aspect, R¹ and R³ are a substituted heteroalkylene linking group containing three carbons, an oxygen or a nitrogen atom and substituted with a halogen. In a preferred embodiment of this aspect, the halogen is a fluorine. In a further aspect, R¹ and R^{b 3} are a substituted heteroalkylene linking group containing three carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups.

In one aspect, the precursor has Formula II-B:

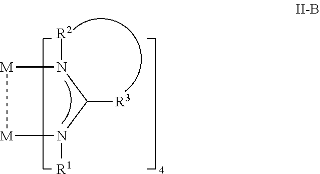

where: M is one of chromium, molybdenum and tungsten;
   R¹ is selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen, a C₃-C₈ aromatic group substituted with an amino group; and
   R² and R³ constitute part of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen.

As those this skilled in the art will understand, the backbone of the alkylenes and heteroalkylenes described in the embodiment above will contain either three or four atoms exclusive of any sub stituents or pendent chains thereon.

In one aspect of this embodiment, R² and R³ constitute part of a 5-member heterocyclic ring. In a further aspect, R² and R³ are an unsubstituted alkylene linking group containing three carbons. In a further aspect, R² and R³ are a substituted alkylene linking group containing three carbons. In a further aspect, R² and R³ are a substituted alkylene linking group containing three carbons substituted by at least one halogen atom. In a further aspect, R² and R³ are a substituted alkylene linking group containing three carbons substituted by at least one fluorine atom. In a further aspect, R² and R³ are a substituted alkylene linking group containing three carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, R² and R³ are an unsubstituted heteroalkylene linking group containing two carbons and an oxygen. In a further aspect, R² and R³ are an unsubstituted heteroalkylene linking group containing two carbons and a nitrogen. In a further aspect, R² and R³ are a substituted heteroalkylene linking group containing two carbons, an oxygen or a nitrogen atom and substituted with a halogen. In a preferred embodiment of this aspect, the halogen is a fluorine. In a further aspect, R² and R³ are a substituted heteroalkylene linking group containing two carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups.

In one aspect of this embodiment, R² and R³ constitute part of a 6-member heterocyclic ring. In a further aspect, R² and R³ are an unsubstituted alkylene linking group containing four carbons. In a further aspect, R² and R³ are a substituted alkylene linking group containing four carbons. In a further aspect, R² and R³ are a substituted alkylene linking group containing four carbons substituted by at least one halogen atom. In a further aspect, R² and R³ are a substituted alkylene linking group containing four carbons substituted by at least one fluorine atom. In a further aspect, R² and R³ are a substituted alkylene linking group containing four carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, R² and R³ are an unsubstituted heteroalkylene linking group containing three carbons and an oxygen. In a further aspect, R² and R³ are an unsubstituted heteroalkylene linking group containing three carbons and a nitrogen. In a further aspect, R² and R³ are a substituted heteroalkylene linking group containing three carbons, an oxygen or a nitrogen atom and substituted with a halogen. In a preferred embodiment of this aspect, the halogen is a fluorine. In a further aspect, R² and R³ are a substituted heteroalkylene linking group containing three carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups.

In one aspect, the precursor has Formula II-C:

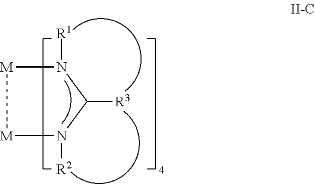

where: M is one of chromium, molybdenum and tungsten; and
   each of (a) R¹ and R³ and (b) R² and R³ independently constitute parts of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen.

As those this skilled in the art will understand, the backbone of the alkylenes and heteroalkylenes described in the embodiment above will contain either three or four atoms exclusive of any sub stituents or pendent chains thereon.

In one aspect of this embodiment, each of (a) R¹ and R³ and (b) R² and R³ each independently constitute part of a 5-member heterocyclic ring. In a further aspect, each of (a) le and R³ and (b) R² and R³ are each independently an unsubstituted alkylene linking group containing three carbons. In a further aspect, each of (a) R¹ and R³ and (b) R² and R³ are each independently a substituted alkylene linking group containing three carbons. In a further aspect, each of (a) R¹ and R³ and (b) R² and R³ are each independently a substituted alkylene linking group containing three carbons substituted by at least one halogen atom. In a further aspect, each of (a) R¹ and R³ and (b) R² and R³ are each independently a substituted alkylene linking group containing three carbons substituted by at least one fluorine atom. In a further aspect, each of (a) R¹ and R³ and (b) R² and R³ are each independently a substituted alkylene linking group containing three carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, each of (a) R¹ and R³ and (b) R² and R³ are each independently an unsubstituted heteroalkylene linking group containing two carbons and an oxygen. In a further aspect, each of (a) R¹ and R³ and (b) R² and R³ are each independently an unsubstituted heteroalkylene linking group containing two carbons and a nitrogen. In a further aspect, each of (a) R¹ and R³ and (b) R² and R³ are each independently a substituted heteroalkylene linking group containing two carbons, an oxygen or a nitrogen atom and substituted with a halogen. In a preferred embodiment of this aspect, the halogen is a fluorine. In a further aspect, each of (a) R¹ and R³ and (b) R² and R³ are each independently a substituted heteroalkylene linking group containing two carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect of this embodiment, each of (a) R¹ and R³ and (b) R² and R³ are the same. In a further aspect of this embodiment, each of (a) R¹ and R³ and (b) R² and R³ are different.

In one aspect of this embodiment, each of (a) R¹ and R³ and (b) R² and R³ each independently constitute part of a 6-member heterocyclic ring. In a further aspect, each of (a) R¹ and R³ and (b) R² and R³ are each independently an unsubstituted alkylene linking group containing four carbons. In a further aspect, each of (a) R¹ and R³ and (b) R² and R³ are each independently a substituted alkylene linking group containing four carbons. In a further aspect, each of (a) R¹ and R³ and (b) R² and R³ are each independently a substituted alkylene linking group containing four carbons substituted by at least one halogen atom. In a further aspect, each of (a) R¹ and R³ and (b) R² and R³ are each independently a substituted alkylene linking group containing four carbons substituted by at least one fluorine atom. In a further aspect, each of (a) R¹ and R³ and (b) R² and R³ are each independently a substituted alkylene linking group containing four carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, each of (a) R¹ and R³ and (b) R² and R³ are each independently an unsubstituted heteroalkylene linking group containing three carbons and an oxygen. In a further aspect, each of (a) R¹ and R³ and (b) R² and R³ are each independently an unsubstituted heteroalkylene linking group containing three carbons and a nitrogen. In a further aspect, each of (a) R¹ and R³ and (b) R² and R³ are each independently a substituted heteroalkylene linking group containing three carbons, an oxygen or a nitrogen atom and substituted with a halogen. In a preferred embodiment of this aspect, the halogen is a fluorine. In a further aspect, each of (a) R¹ and R³ and (b) R² and R³ are each independently a substituted heteroalkylene linking group containing three carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect of this embodiment, each of (a) R¹ and R³ and (b) R² and R³ are the same. In a further aspect of this embodiment, each of (a) R¹ and R³ and (b) R² and R³ are different.

In some embodiments, the tethered heterocyclic Ad ligands (Formula II-A and Formula II-B) and/or heterocyclic bicyclic Ad ligands (Formula II-C) have a structure as illustrated in Table 2:

TABLE 2
Amidinate Ligands

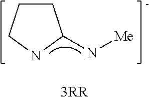

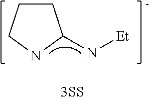

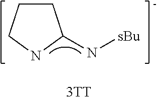

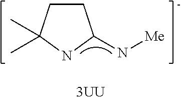

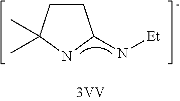

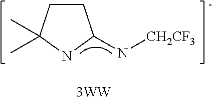

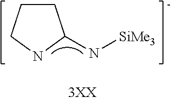

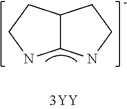

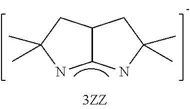

In one aspect of this embodiment, the compounds of Formula II-A and/or II-B have the following structure where the Ad ligand is an iminopyrrolidinate ligand:

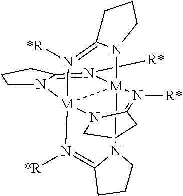

where: M=Cr, Mo, W; and
   R*=whichever of R¹ and R² does not form a ring with R³ and is a C₁-C₅ substituted or unsubstituted alky group.

In another aspect of this embodiment, the compounds of Formula II-A and/or II-B have the following structure where the Ad ligand is an iminopiperidinate ligand:

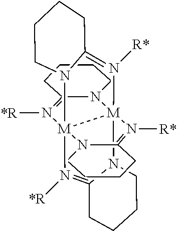

where: M=Cr, Mo, W; and
   R*=whichever of R¹ and R² does not form a ring with R³ and is a C₁-C₅ substituted or unsubstituted alkyl group.

In one example of this embodiment, M=Mo and R* is sec-butyl group (—CH(CH₃)CH₂CH₃):

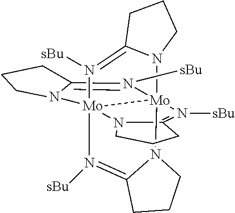

In one aspect, the precursor has Formula II-D:

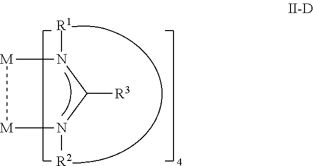

where: M is one of chromium, molybdenum and tungsten; and
   each of (a) R¹ and R² independently constitute parts of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen.

As those this skilled in the art will understand, the backbone of the alkylenes and heteroalkylenes described in the embodiment above will contain either two or three atoms exclusive of any sub stituents or pendent chains thereon.

In some embodiments, the tethered heterocyclic Ad ligands (Formula II-D) have a structure as illustrated in Table 3, and are based on 2-imidazoline ligand:

TABLE 3
Amidinate Ligands (based on 2-imidazoline ligand)

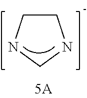

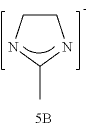

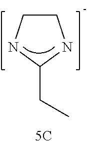

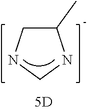

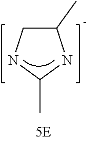

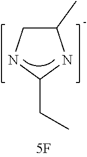

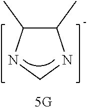

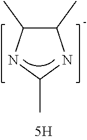

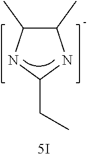

In some embodiments, the tethered heterocyclic Ad ligands (Formula II-D) have a structure as illustrated in Table 4, and are based on 1,4,5,6 tetrahydropyrimidine ligand:

TABLE 4
Amidinate Ligands (based on 1,4,5,6-tetrahydropyrimidine ligand)

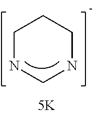

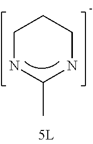

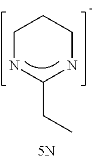

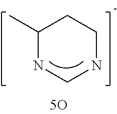

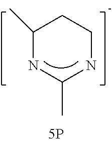

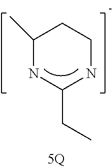

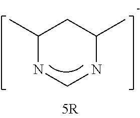

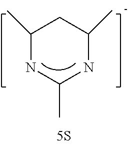

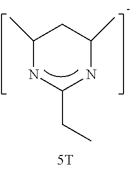

In one aspect of this embodiment, the compounds of Formula II-D have the following structure where the Ad ligand is 2-methyl-2-imidazoline ligand:

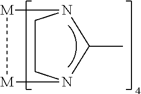

where: M=Cr, Mo, W.

In one aspect of this embodiment, the compounds of Formula II-D have the following structure where the Ad ligand is an 1,4,5,6-tetrahydropyrimidine ligand:

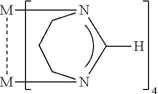

where: M=Cr, Mo, W.

### Guanidinate Paddlewheel Precursors

Another aspect of the disclosed and claimed subject matter pertains to guanidinate (“Gd’) paddlewheel compounds of chromium, molybdenum and tungsten of Formula III:

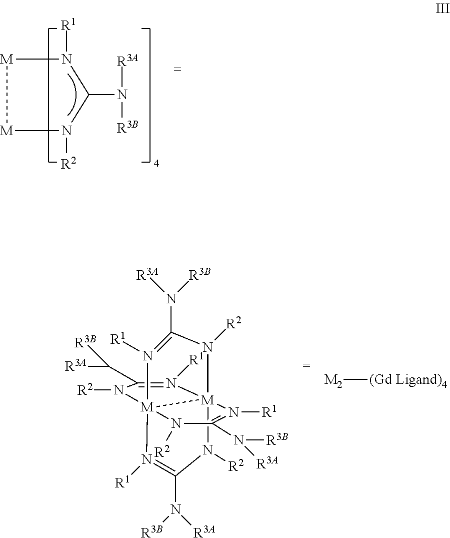

where: M is one of chromium, molybdenum and tungsten; and
   R¹, R², R^{3A} and R^{3B} are each independently selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen and a C₃-C₈ aromatic group substituted with an amino group. In one aspect of this embodiment, all four guanidinate ligands have the same chemical structure. In another aspect of this embodiment, two or more of the guanidinate ligands have the same chemical structure. In another aspect of this embodiment, all four guanidinate ligands have a different chemical structure.

In one aspect of this embodiment, R¹, R² and R³ are each independently selected from H, unsubstituted linear C₁ to C₃ alkyl group and an unsubstituted branched C₃ or C₄ alkyl group. In one aspect, one or more of R¹, R² and R³ is a methyl group. In one aspect, one or more of R¹, R² and R³ is a ethyl group. In one aspect, one or more of R¹, R² and R³ is a propyl group. In one aspect, one or more of R¹, R² and R³ is an isopropyl group. In one aspect, one or more of R¹, R² and R³ is a sec-butyl group. In one aspect, one or more of R¹, R² and R³ is a n-butyl group. In one aspect, one or more of R¹, R² and R³ is an iso-butyl group.

In one aspect of this embodiment, M is chromium. In another aspect of this embodiment, M is molybdenum. In another aspect of this embodiment, M is tungsten.

In some embodiments, the tethered guanidinate ligand (“Gd Ligand”) has a structure as illustrated in Table 5:

TABLE 5
Guanidinate Ligands

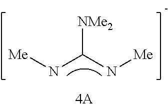

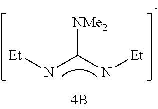

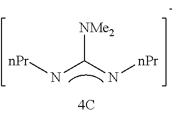

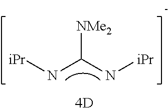

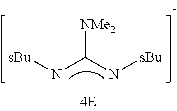

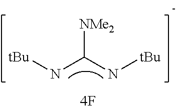

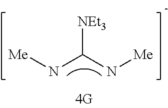

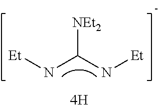

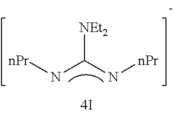

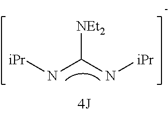

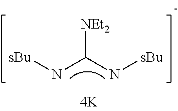

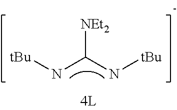

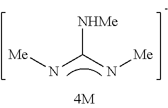

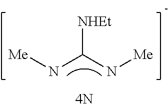

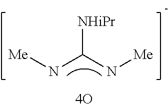

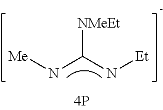

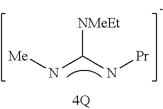

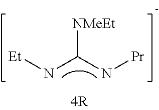

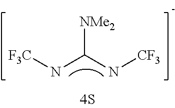

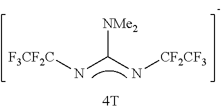

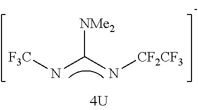

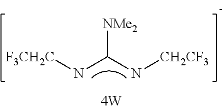

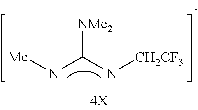

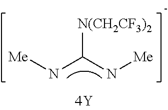

In additional aspects of this embodiment, the compounds of Formula III include heterocyclic Gd ligands (Formula IV-A and Formula IV-B) and/or heterocyclic bicyclic Ad ligands (Formula IV-C) as shown below where one or both of (a) R¹ and R^{3A or 3B} and (b) R² and R^{3A or 3B} independently constitute parts of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen.

In one aspect, the precursor has Formula IV-A:

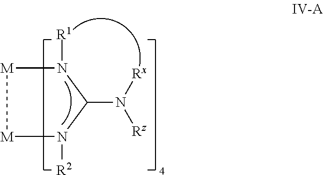

where: M is one of chromium, molybdenum and tungsten;
   R² is selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen, a C₃-C₈ aromatic group substituted with an amino group; and
   R¹ and R^{X} constitute part of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen, where R^{Z} is one of R^{3A} and R^{3B} and R^{X} is the other of R^{3A} and R^{3B} that is not connected by a linking group to R¹.

As those this skilled in the art will understand, the backbone of the alkylenes and heteroalkylenes described in the embodiment above will contain either three or four atoms exclusive of any sub stituents or pendent chains thereon.

In one aspect of this embodiment, R¹ and R^{X} constitute part of a 5-member heterocyclic ring. In a further aspect, R¹ and R^{X} are an unsubstituted alkylene linking group containing three carbons. In a further aspect, R¹ and R^{X} are a substituted alkylene linking group containing three carbons. In a further aspect, R¹ and R^{X} are a substituted alkylene linking group containing three carbons substituted by at least one halogen atom. In a further aspect, R¹ and R^{X} are a substituted alkylene linking group containing three carbons substituted by at least one fluorine atom. In a further aspect, R¹ and R^{X} are a substituted alkylene linking group containing three carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, R¹ and R^{X} are an unsubstituted heteroalkylene linking group containing two carbons and an oxygen. In a further aspect, R¹ and R^{X} are an unsubstituted heteroalkylene linking group containing two carbons and a nitrogen. In a further aspect, R¹ and R^{X} are a substituted heteroalkylene linking group containing two carbons, an oxygen or a nitrogen atom and substituted with a halogen. In a preferred embodiment of this aspect, the halogen is a fluorine. In a further aspect, R¹ and R^{X} are a substituted heteroalkylene linking group containing two carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups.

In one aspect of this embodiment, R¹ and R^{X} constitute part of a 6-member heterocyclic ring. In a further aspect, R¹ and R^{X} are an unsubstituted alkylene linking group containing four carbons. In a further aspect, R¹ and R^{X} are a substituted alkylene linking group containing four carbons. In a further aspect, R¹ and R^{X} are a substituted alkylene linking group containing four carbons substituted by at least one halogen atom. In a further aspect, R¹ and R^{X} are a substituted alkylene linking group containing four carbons substituted by at least one fluorine atom. In a further aspect, R¹ and R^{X} are a substituted alkylene linking group containing four carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, R¹ and R^{X} are an unsubstituted heteroalkylene linking group containing three carbons and an oxygen. In a further aspect, R¹ and R^{X} are an unsubstituted heteroalkylene linking group containing three carbons and a nitrogen. In a further aspect, R¹ and R^{X} are a substituted heteroalkylene linking group containing three carbons, an oxygen or a nitrogen atom and substituted with a halogen. In a preferred embodiment of this aspect, the halogen is a fluorine. In a further aspect, R¹ and R^{X} are a substituted heteroalkylene linking group containing three carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or mor more methyl groups.

In one aspect, the precursor has Formula IV-B:

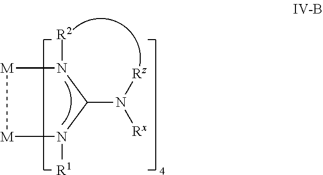

where: M is one of chromium, molybdenum and tungsten;
   R¹ is selected from H, D, an unsubstituted linear C₁-C₆ alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)_{3,} a C₃-C₈ unsubstituted cyclic alkyl group, a C₃-C₈ cyclic alkyl group substituted with a halogen, a C₃-C₈ cyclic alkyl group substituted with an amino group, a C³-C⁸ unsubstituted aromatic group, a C₃-C₈ aromatic group substituted with a halogen, a C₃-C₈ aromatic group substituted with an amino group; and
   R² and R^{Z} constitute part of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen, where R^{Z} is one of R^{3A} and R^{3B} and R^{X} is the other of R^{3A} and R^{3B} that is not connected by a linking group to R².

As those this skilled in the art will understand, the backbone of the alkylenes and heteroalkylenes described in the embodiment above will contain either three or four atoms exclusive of any sub stituents or pendent chains thereon.

In one aspect of this embodiment, R² and R^{Z} constitute part of a 5-member heterocyclic ring. In a further aspect, R² and R^{Z} are an unsubstituted alkylene linking group containing three carbons. In a further aspect, R² and R^{Z} are a substituted alkylene linking group containing three carbons. In a further aspect, R² and R^{Z} are a substituted alkylene linking group containing three carbons substituted by at least one halogen atom. In a further aspect, R² and R^{Z} are a substituted alkylene linking group containing three carbons substituted by at least one fluorine atom. In a further aspect, R² and R^{Z} are a substituted alkylene linking group containing three carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, R² and R³ are an unsubstituted heteroalkylene linking group containing two carbons and an oxygen. In a further aspect, R² and R^{Z} are an unsubstituted heteroalkylene linking group containing two carbons and a nitrogen. In a further aspect, R² and R^{Z} are a substituted heteroalkylene linking group containing two carbons, an oxygen or a nitrogen atom and substituted with a halogen. In a preferred embodiment of this aspect, the halogen is a fluorine. In a further aspect, R² and R^{Z} are a substituted heteroalkylene linking group containing two carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups.

In one aspect of this embodiment, R² and R^{Z} constitute part of a 6-member heterocyclic ring. In a further aspect, R² and R^{Z} are an unsubstituted alkylene linking group containing four carbons. In a further aspect, R² and R^{Z} are a substituted alkylene linking group containing four carbons. In a further aspect, R² and R^{Z} are a substituted alkylene linking group containing four carbons substituted by at least one halogen atom. In a further aspect, R² and R^{Z} are a substituted alkylene linking group containing four carbons substituted by at least one fluorine atom. In a further aspect, R² and R^{Z} are a substituted alkylene linking group containing four carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, R² and R^{Z} are an unsubstituted heteroalkylene linking group containing three carbons and an oxygen. In a further aspect, R² and R^{Z} are an unsubstituted heteroalkylene linking group containing three carbons and a nitrogen. In a further aspect, R² and R^{Z} are a substituted heteroalkylene linking group containing three carbons, an oxygen or a nitrogen atom and substituted with a halogen. In a preferred embodiment of this aspect, the halogen is a fluorine. In a further aspect, R² and R^{Z} are a substituted heteroalkylene linking group containing three carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups.

In one aspect, the precursor has Formula IV-C:

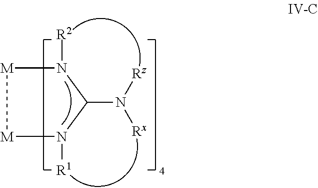

where: M is one of chromium, molybdenum and tungsten; and
   each of (a) R¹ and R^{X} and (b) R² and R^{Z} independently constitute parts of a 5- or 6-member heterocyclic ring and are one of (i) an unsubstituted alkylene linking group, (ii) a substituted alkylene linking group, (iii) an unsubstituted heteroalkylene linking group where containing a hetero atom is selected from oxygen and nitrogen and (iv) a substituted heteroalkylene linking group containing a hetero atom selected from oxygen and nitrogen, where R¹ is one of R^{3A} and R^{3B} and R^{X} is the other of R^{3A} and R^{3B}.

As those this skilled in the art will understand, the backbone of the alkylenes and heteroalkylenes described in the embodiment above will contain either three or four atoms exclusive of any sub stituents or pendent chains thereon.

In one aspect of this embodiment, each of (a) R¹ and R^{X} and (b) R² and R^{Z} each independently constitute part of a 5-member heterocyclic ring. In a further aspect, each of (a) R¹ and R^{X} and (b) R² and R¹ are each independently an unsubstituted alkylene linking group containing three carbons. In a further aspect, each of (a) R¹ and R^{X} and (b) R² and R^{Z} are each independently a substituted alkylene linking group containing three carbons. In a further aspect, each of (a) R¹ and R^{X} and (b) R² and R¹ are each independently a substituted alkylene linking group containing three carbons substituted by at least one halogen atom. In a further aspect, each of (a) R¹ and R^{X} and (b) R² and R^{Z} are each independently a substituted alkylene linking group containing three carbons substituted by at least one fluorine atom. In a further aspect, each of (a) R¹ and R^{X} and (b) R² and R^{Z} are each independently a substituted alkylene linking group containing three carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, each of (a) R¹ and R^{X} and (b) R² and R^{Z} are each independently an unsubstituted heteroalkylene linking group containing two carbons and an oxygen. In a further aspect, each of (a) R¹ and R^{X} and (b) R² and R¹ are each independently an unsubstituted heteroalkylene linking group containing two carbons and a nitrogen. In a further aspect, each of (a) R¹ and R^{X} and (b) R² and R^{Z} are each independently a substituted heteroalkylene linking group containing two carbons, an oxygen or a nitrogen atom and substituted with a halogen. In a preferred embodiment of this aspect, the halogen is a fluorine. In a further aspect, each of (a) R¹ and R^{X} and (b) R² and R^{Z} are each independently a substituted heteroalkylene linking group containing two carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect of this embodiment, each of (a) R¹ and R^{X} and (b) R² and R^{Z} are the same. In a further aspect of this embodiment, each of (a) R¹ and R^{X} and (b) R² and R^{Z} are different.

In one aspect of this embodiment, each of (a) R¹ and R^{X} and (b) R² and R^{Z} each independently constitute part of a 6-member heterocyclic ring. In a further aspect, each of (a) R¹ and R^{X} and (b) R² and R^{Z} are each independently an unsubstituted alkylene linking group containing four carbons. In a further aspect, each of (a) R¹ and R^{X} and (b) R² and R^{Z} are each independently a substituted alkylene linking group containing four carbons. In a further aspect, each of (a) R¹ and R^{X} and (b) R² and R^{Z} are each independently a substituted alkylene linking group containing four carbons substituted by at least one halogen atom. In a further aspect, each of (a) R¹ and R^{X} and (b) R² and R^{Z} are each independently a substituted alkylene linking group containing four carbons substituted by at least one fluorine atom. In a further aspect, each of (a) R¹ and R^{X} and (b) R² and R¹ are each independently a substituted alkylene linking group containing four carbons substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect, each of (a) R¹ and R^{X} and (b) R² and R^{Z} are each independently an unsubstituted heteroalkylene linking group containing three carbons and an oxygen. In a further aspect, each of (a) R¹ and R^{X} and (b) R² and R^{Z} are each independently an unsubstituted heteroalkylene linking group containing three carbons and a nitrogen. In a further aspect, each of (a) R¹ and R^{X} and (b) R² and R^{Z} are each independently a substituted heteroalkylene linking group containing three carbons, an oxygen or a nitrogen atom and substituted with a halogen. In a preferred embodiment of this aspect, the halogen is a fluorine. In a further aspect, each of (a) R¹ and R^{X} and (b) R² and R^{Z} are each independently a substituted heteroalkylene linking group containing three carbons, an oxygen or a nitrogen atom and substituted by at least one of a methyl group, ethyl group, n-propyl group, isopropyl, s-butyl, n-butyl, isobutyl or t-butyl group. In a preferred embodiment of this aspect, the substituent is one or more methyl groups. In a further aspect of this embodiment, each of (a) R¹ and R^{X} and (b) R² and R^{Z} are the same. In a further aspect of this embodiment, each of (a) R¹ and R^{X} and (b) R² and R^{Z} are different.

In some embodiments, the tethered heterocyclic Gd ligands (Formula IV-A and Formula IV-B) and/or heterocyclic bicyclic Gd ligands. (Formula IV-C) have a structure as illustrated in Table 6:

TABLE 6
Guanidinate Ligands

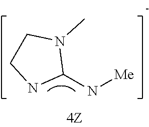

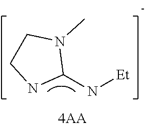

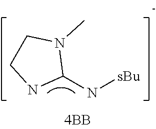

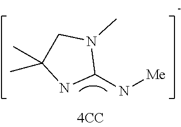

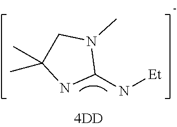

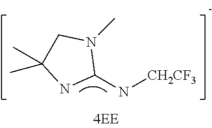

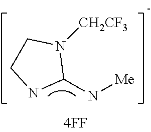

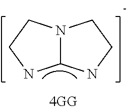

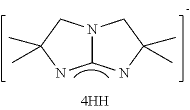

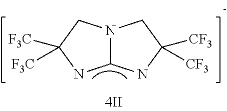

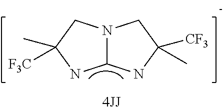

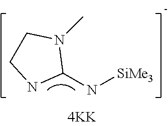

### Highlighted Paddlewheel Precursors

Table 7 below identifies specific embodiments of paddlewheel precursors of general formulae (i) M₂-(Ad Ligand)₄ and (ii) M₂-(Gd Ligand)₄ that include the ligands set forth in Tables 1-6.

TABLE 7

| Formula | Compound |
|---|---|
| M₂-(3A)₄ | 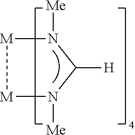 |
| M₂-(3B)₄ | 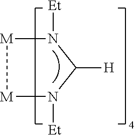 |
| M₂-(3D)₄ | 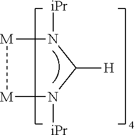 |
| M₂-(3TT)₄ | 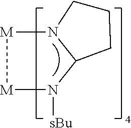 |
| M₂-(3XX)₄ | 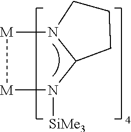 |
| M₂-(3UU)₄ | 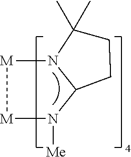 |
| M₂-(3RR)₄ | 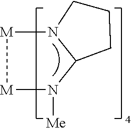 |
| M₂-(4Z)₄ | 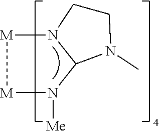 |
| M₂-(3G)₄ | 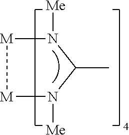 |
| M₂-(3H)₄ | 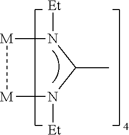 |
| M₂-(3Z)₄ | 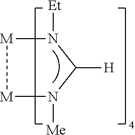 |
| M₂-(4A)₄ | 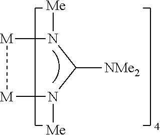 |
| M₂-(3KK)₄ | 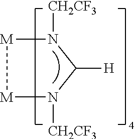 |
| M₂-(3MM)₄ | 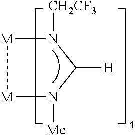 |
| M₂-(3QQ)₄ | 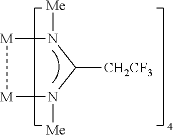 |
| M₂-(3YY)₄ | 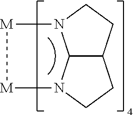 |
| M₂-(4GG)₄ | 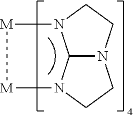 |
| M₂-(4HH)₄ | 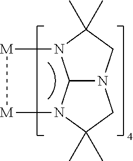 |
| M₂-(5K)₄ | 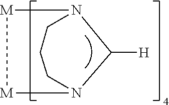 |
| M₂-(5B)₄ | 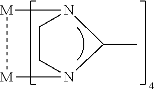 |

In one preferred embodiment, the precursor is M₂-(3A)₄ set forth in Table 7 where M=Mo:

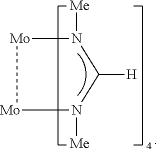

In one preferred embodiment, the precursor is M₂-(3A)₄ set forth in Table 7 where M=Cr:

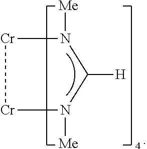

In one preferred embodiment, the precursor is M₂-(3A)₄ set forth in Table 7 where M=W:

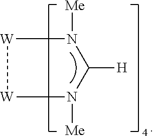

In another preferred embodiment, the precursor is M₂-(3B)₄ set forth in Table 7 where M=Mo:

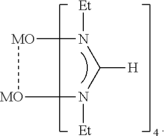

In another preferred embodiment, the precursor is M₂-(3B)₄ set forth in Table 7 where M=Cr:

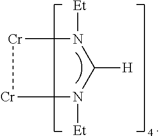

In another preferred embodiment, the precursor is M₂-(3B)₄ set forth in Table 7 where M=W:

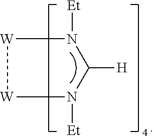

In one preferred embodiment, the precursor is M₂-(3D)₄ set forth in Table 7 where M=W:

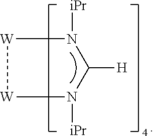

In one preferred embodiment, the precursor is M₂-(3XX)₄ set forth in Table 7 where M=Mo:

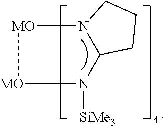

In one preferred embodiment, the precursor is M₂-(3XX)₄ set forth in Table 7 where M=Cr:

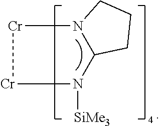

In one preferred embodiment, the precursor is M₂-(3XX)₄ set forth in Table 7 where M=W:

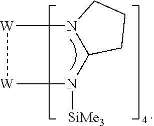

In one preferred embodiment, the precursor is M₂-(3UU)₄ set forth in Table 7 where M=Mo:

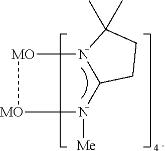

In one preferred embodiment, the precursor is M₂-(3UU)₄ set forth in Table 7 where M=Cr:

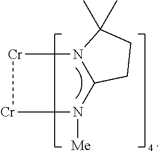

In one preferred embodiment, the precursor is M2-(3UU)4 set forth in Table 7 where M=W:

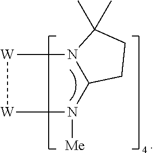

In one preferred embodiment, the precursor is M2-(3Z)4 set forth in Table 7 where M=Mo:

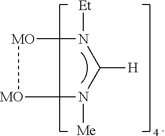

In one preferred embodiment, the precursor is M2-(3Z)4 set forth in Table 7 where M=Cr:

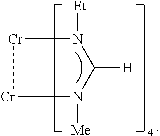

In one preferred embodiment, the precursor is M2-(3Z)4 set forth in Table 7 where M=W:

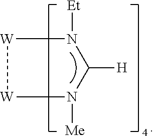

In one preferred embodiment, the precursor is M2-(3Z)4 set forth in Table 7 where M=Mo:

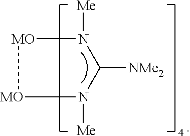

In one preferred embodiment, the precursor is M2-(3Z)4 set forth in Table 7 where M=Cr:

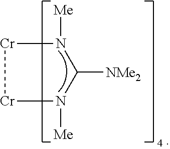

In one preferred embodiment, the precursor is M₂-(3Z)₄ set forth in Table 7 where M=W:

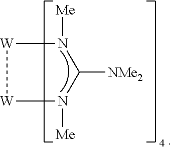

In one preferred embodiment, the precursor is M₂-(3KK)₄ set forth in Table 7 where M=Mo:

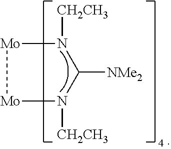

In one preferred embodiment, the precursor is M₂-(3KK)₄ set forth in Table 7 where M=Cr:

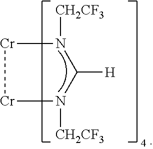

In one preferred embodiment, the precursor is M₂-(3KK)₄ set forth in Table 7 where M=W:

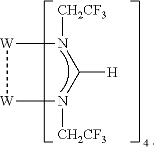

In one preferred embodiment, the precursor is M2-(3QQ)4 set forth in Table 7 where M=Mo:

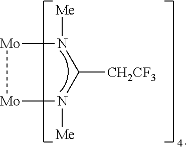

In one preferred embodiment, the precursor is M2-(3QQ)4 set forth in Table 7 where M=Cr:

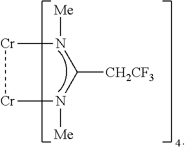

In one preferred embodiment, the precursor is M2-(3QQ)4 set forth in Table 7 where M=W:

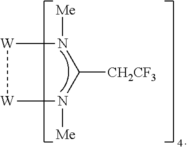

In one preferred embodiment, the precursor is M2-(3TT)4 set forth in Table 7 where M=Mo:

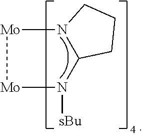

In one preferred embodiment, the precursor is M2-(3TT)4 set forth in Table 7 where M=Cr:

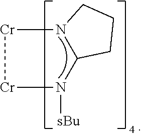

In one preferred embodiment, the precursor is M2-(3TT)4 set forth in Table 7 where M=W:

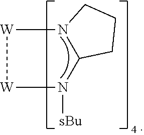

The disclosed and claimed precursors are not limited to those exemplified in Table 7. In addition, the Ad ligands and Gd ligands are not limited to those exemplified in Tables 1-7.

### Method of Synthesis

The disclosed and claimed paddlewheel precursors are generally made according to the following formula (which is exemplified here using molybdenum to form a molybdenum (II) amidinate paddlewheel compound):

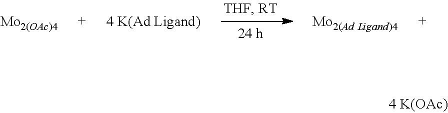

In the above reaction, molybdenum (II) acetate is suspended in a suitable solvent (e.g., THF, toluene, hexane) and a solution of potassium amidinate is slowly added. Potassium amidinate can be prepared by reaction of the amidinium sulphates with potassium hexamethyldisilazide. The reaction mixture is stirred for a period of time (ca. 4-48 h) after which the solvent is removed by vacuum distillation. The crude reaction material is extracted with a suitable solvent (e.g., hexane, toluene, THF) and separated from any insoluble solid by filtration. The solvent of the filtrate solution is removed by vacuum distillation to afford the product as a solid. The solid is purified by vacuum sublimation.

In an alternative synthesis route the disclosed and claimed paddlewheel precursors are generally made according to the following formula (which is exemplified here using molybdenum to form a molybdenum (II) amidinate paddlewheel compound):

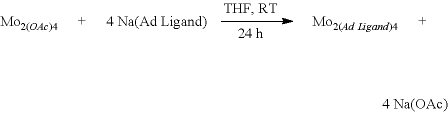

In the above reaction, molybdenum (II) acetate is suspended in a suitable solvent (e.g., THF, toluene, hexane) and a solution of sodium amidinate is slowly added. Sodium amidinate can be prepared by reaction of “amidine” (protonated amidinate ligand) with sodium hydride. The reaction mixture is stirred for a period of time (ca. 4-48 h) after which the solvent is removed by vacuum distillation. The crude reaction material is extracted with a suitable solvent (e.g., hexane, toluene, THF) and separated from any insoluble solid by filtration. The solvent of the filtrate solution is removed by vacuum distillation to afford the product as a solid. The solid is purified by re-crystallization.

Those skilled in the art will recognize that other metals (i.e., chromium and tungsten) and/or the guanidinate ligands can be used in the same general process. Thus, in one embodiment, the disclosed and claimed subject matter includes synthesizing precursors of formula M₂-(Ad Ligand)₄ and/or M₂-(Ad Ligand)₄ according to the following reaction

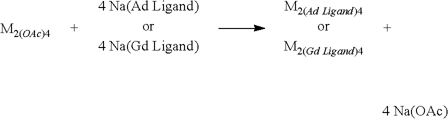

where M is one of chromium, molybdenum and tungsten and the Ad Ligand and Gd ligand are as described above (including in Tables 1-6). In one aspect of this embodiment M is chromium. In one aspect of this embodiment M is molybdenum. In one aspect of this embodiment M is tungsten. In a further aspect of this embodiment, the precursors of formula M₂-(Ad Ligand)₄ and/or M₂-(Ad Ligand)₄ synthesized by this process include those set forth in Table 7.

### Method of Use

The disclosed precursors may be deposited to form chromium, molybdenum and tungsten containing films using any chemical vapor deposition process known to those of skill in the art. As used herein, the term “chemical vapor deposition process” refers to any process wherein a substrate is exposed to one or more volatile precursors, which react and/or decompose on the substrate surface to produce the desired deposition. As used herein, the term “atomic layer deposition process” refers to a self-limiting (e.g., the amount of film material deposited in each reaction cycle is constant), sequential surface chemistry that deposits films of materials onto substrates of varying compositions. Although the precursors, reagents and sources used herein may be sometimes described as “gaseous,” it is understood that the precursors can be either liquid or solid which are transported with or without an inert gas into the reactor via direct vaporization, bubbling or sublimation. In some case, the vaporized precursors can pass through a plasma generator. The term “reactor” as used herein, includes without limitation, reaction chamber, reaction vessel or deposition chamber.

Chemical vapor deposition processes in which the disclosed and claimed precursors can be utilized include, but are not limited to, those used for the manufacture of semiconductor type microelectronic devices such as ALD, CVD, pulsed CVD, plasma enhanced ALD (PEALD) and/or plasma enhanced CVD (PECVD). Examples of suitable deposition processes for the method disclosed herein include, but are not limited to, cyclic CVD (CCVD), MOCVD (Metal Organic CVD), thermal chemical vapor deposition, plasma enhanced chemical vapor deposition (“PECVD”), high density PECVD, photon assisted CVD, plasma-photon assisted (“PPECVD”), cryogenic chemical vapor deposition, chemical assisted vapor deposition, hot-filament chemical vapor deposition, CVD of a liquid polymer precursor, deposition from supercritical fluids, and low energy CVD (LECVD). In certain embodiments, the metal containing films are deposited via atomic layer deposition (ALD), plasma enhanced ALD (PEALD) or plasma enhanced cyclic CVD (PECCVD) process.

In one embodiment, for example, the metal-containing film is deposited using an ALD process. In another embodiment, the metal-containing film is deposited using a CCVD process. In a further embodiment, the metal-containing film is deposited using a thermal CVD process.

Suitable substrates on which the disclosed and claimed precursors can be deposited are not particularly limited and vary depending on the final use intended. For example, the substrate may be chosen from oxides such as HfO₂ based materials, TiO₂ based materials, ZrO₂ based materials, rare earth oxide-based materials, ternary oxide-based materials, etc. or from nitride-based films. Other substrates may include solid substrates such as metal substrates (for example, Au, Pd, Rh, Ru, W, Al, Ni, Ti, Co, Pt and metal silicides (e.g., TiSi₂, CoSi₂, and NiSi₂); metal nitride containing substrates (e.g., TaN, TiN, WN, TaCN, TiCN, TaSiN, and TiSiN); semiconductor materials (e.g., Si, SiGe, GaAs, InP, diamond, GaN, and SiC); insulators (e.g., SiO₂, Si₃N₄, SiON, HfO₂, Ta₂O₅, ZrO₂, TiO₂, Al₂O₃, and barium strontium titanate); combinations thereof.

Preferred substrates include silicon oxide, aluminum oxide, TiN, Ru, Co, Cu and Si type substrates. One advantage of these precursors is the ability to deposit thin continuous films directly on silicon and aluminum oxides.

In such deposition methods and processes an oxidizing agent can be utilized. The oxidizing agent is typically introduced in gaseous form. Examples of suitable oxidizing agents include, but are not limited to, oxygen gas, water vapor, ozone, oxygen plasma, or mixtures thereof

The deposition methods and processes may also involve one or more purge gases. The purge gas, which is used to purge away unconsumed reactants and/or reaction byproducts, is an inert gas that does not react with the precursors. Exemplary purge gases include, but are not limited to, argon (Ar), nitrogen (N₂), helium (He), neon, and mixtures thereof. For example, a purge gas such as Ar is supplied into the reactor at a flow rate ranging from about 10 to about 2000 sccm for about 0.1 to 10000 seconds, thereby purging the unreacted material and any byproduct that may remain in the reactor.

The deposition methods and processes require that energy be applied to the at least one of the precursors, oxidizing agent, other precursors or combination thereof to induce reaction and to form the metal-containing film or coating on the substrate. Such energy can be provided by, but not limited to, thermal, plasma, pulsed plasma, helicon plasma, high density plasma, inductively coupled plasma, X-ray, e-beam, photon, remote plasma methods, and combinations thereof. In some processes, a secondary RF frequency source can be used to modify the plasma characteristics at the substrate surface. When utilizing plasma, the plasma-generated process may include a direct plasma-generated process in which plasma is directly generated in the reactor, or alternatively a remote plasma-generated process in which plasma is generated outside of the reactor and supplied into the reactor.

When utilized in such deposition methods and processes suitable precursors—such as those presently disclosed and claimed—may be delivered to the reaction chamber such as a CVD or ALD reactor in a variety of ways. In some instances, a liquid delivery system may be utilized. In other instances, a combined liquid delivery and flash vaporization process unit may be employed, such as, for example, the turbo vaporizer manufactured by MSP Corporation of Shoreview, MN, to enable low volatility materials to be volumetrically delivered, which leads to reproducible transport and deposition without thermal decomposition of the precursor. The precursor compositions described herein can be effectively used as source reagents via direct liquid injection (DLI) to provide a vapor stream of these metal precursors into an ALD or CVD reactor.

When used in these deposition methods and processes, the disclosed and claimed precursors include hydrocarbon solvents which are particularly desirable due to their ability to be dried to sub-ppm levels of water. Exemplary hydrocarbon solvents that can be used in the precursors include, but are not limited to, toluene, mesitylene, cumene (isopropylbenzene), p-cymene (4-isopropyl toluene), 1,3-diisopropylbenzene, octane, dodecane, 1,2,4-trimethylcyclohexane, n-butylcyclohexane, and decahydronaphthalene (decalin). The disclosed and claimed precursors can also be stored and used in stainless steel containers. In certain embodiments, the hydrocarbon solvent is a high boiling point solvent or has a boiling point of 100 degrees Celsius or greater. The disclosed and claimed precursors can also be mixed with other suitable metal precursors, and the mixture used to deliver both metals simultaneously for the growth of a binary metal-containing films.

A flow of argon and/or other gas may be employed as a carrier gas to help deliver a vapor containing at least one of the disclosed and claimed precursors to the reaction chamber during the precursor pulsing. When delivering the precursors, the reaction chamber process pressure is between 1 and 50 torr, preferably between 5 and 20 torr.

Substrate temperature can be an important process variable in the deposition of high-quality metal-containing films. Typical substrate temperatures range from about 150° C. to about 550° C. Higher temperatures can promote higher film growth rates.

In view of the forgoing, those skilled in the art will recognize that the disclosed and claimed subject matter further include the use of the disclosed and claimed precursors in Chemical vapor deposition processes as follows.

In one embodiment, the disclosed and claimed subject matter includes a method for forming a transition metal-containing film on at least one surface of a substrate that includes the steps of:
a. providing the at least one surface of the substrate in a reaction vessel;
b. forming a transition metal-containing film on the at least one surface by a deposition process chosen from a thermal chemical vapor deposition (CVD) process and a thermal atomic layer deposition (ALD) process using one or more of the disclosed and claimed precursors of as a metal source compound for the deposition process.
   In a further aspect of this embodiment, the method includes introducing at least one reactant into the reaction vessel. In a further aspect of this embodiment, the method includes introducing at least one reactant into the reaction vessel where the at least one reactant is selected from the group of water, diatomic oxygen, oxygen plasma, ozone, NO, N₂O, NO₂, carbon monoxide, carbon dioxide and combinations thereof In another aspect of this embodiment, the method includes introducing at least one reactant into the reaction vessel where the at least one reactant is selected from the group of ammonia, hydrazine, monoalkylhydrazine, dialkylhydrazine, nitrogen, nitrogen/hydrogen, ammonia plasma, nitrogen plasma, nitrogen/hydrogen plasma, and combinations thereof. In another aspect of this embodiment, the method includes introducing at least one reactant into the reaction vessel where the at least one reactant is selected from the group hydrogen, hydrogen plasma, a mixture of hydrogen and helium, a mixture of hydrogen and argon, hydrogen/helium plasma, hydrogen/argon plasma, boron-containing compounds, silicon-containing compounds and combinations thereof.

In one embodiment, the disclosed and claimed subject matter includes a method of forming a transition metal-containing film via a thermal atomic layer deposition (ALD) process or thermal ALD-like process that includes the steps of:
a. providing a substrate in a reaction vessel;
b. introducing into the reaction vessel one or more of the disclosed and claimed precursors;
c. purging the reaction vessel with a first purge gas;
d. introducing into the reaction vessel a source gas;
e. purging the reaction vessel with a second purge gas;
f. sequentially repeating steps b through e until a desired thickness of the transition metal-containing film is obtained.
   In a further aspect of this embodiment, the source gas is one or more of an oxygen-containing source gas selected from water, diatomic oxygen, oxygen plasma, ozone, NO, N₂O, NO₂, carbon monoxide, carbon dioxide and combinations thereof In another aspect of this embodiment, the source gas is one or more of a nitrogen-containing source gas selected from ammonia, hydrazine, monoalkylhydrazine, dialkylhydrazine, nitrogen, nitrogen/hydrogen, ammonia plasma, nitrogen plasma, nitrogen/hydrogen plasma and mixture thereof In a further aspect of this embodiment, the method the first and second purge gases are each independently selected one or more of argon, nitrogen, helium, neon, and combinations thereof. In a further aspect of this embodiment, the method further includes applying energy to the at least one of precursor, the source gas, the substrate, and combinations thereof, wherein the energy is one or more of thermal, plasma, pulsed plasma, helicon plasma, high density plasma, inductively coupled plasma, X-ray, e-beam, photon, remote plasma methods and combinations thereof In a further aspect of this embodiment, step b of the method further includes introducing into the reaction vessel the precursor using a stream of carrier gas to deliver a vapor of the precursor into the reaction vessel. In a further aspect of this embodiment, step b of the method further includes use of a solvent medium comprising one or more of toluene, mesitylene, isopropylbenzene, 4-isopropyl toluene, 1,3-diisopropylbenzene, octane, dodecane, 1,2,4-trimethylcyclohexane, n-butylcyclohexane, and decahydronaphthalene and combinations thereof

In one embodiment, the disclosed and claimed precursors are utilized to deposit thin liner or seed layer followed by deposition of bulk metal film using another precursor. One advantage of the precursors of this invention is the ability to deposit low resistivity thin films directly on metal oxide and silicon oxide substrates. Without being bound by theory it is believed that thin films deposited by the precursors of invention may initiate growth of metal films from halogen-containing precursors, may prevent halogen diffusion into the substrate, may reduce stress caused by bulk metal film, and may improve step coverage of metal films. In one embodiment bulk metal film is deposited using halogen-containing precursor. Halogen-containing precursors include but are not limited to molybdenum pentachloride (MoCl₅), molybdenum dioxide dichloride (MoO₂Cl₂), molybdenum hexafluoride (MoF₆), tungsten pentachloride (WCl₅), tungsten hexachloride (WCl₆), tungsten dioxide dichloride (WO₂Cl₂), tungsten hexafluoride (WF₆), vanadium tetrachloride (VCl₄), vanadium oxytrichloride (VOCl₃), etc.

In one embodiment, the disclosed and claimed subject matter includes a method of forming low resistivity transition metal-containing film via an atomic layer deposition (ALD) process or ALD-like process that includes the steps of:
a. forming a transition metal-containing film on the at least one surface by a deposition process chosen from a chemical vapor deposition (CVD) process and an atomic layer deposition (ALD) process using one or more of the disclosed and claimed precursors of as a metal source compound for the deposition process.
b. forming a transition metal-containing film directly on the surface deposited in step (a) by a deposition process chosen from a chemical vapor deposition (CVD) process and an atomic layer deposition (ALD) process using at least one halogen-containing precursors

In one embodiment, the films deposited by the above methods and using the disclosed and claimed precursors have a resistivity below approximately 500 μOhm cm. In another embodiment, the films deposited by the above methods and using the disclosed and claimed precursors have a resistivity below approximately 400 μOhm cm. In another embodiment, the films deposited by the above methods and using the disclosed and claimed precursors have a resistivity below approximately 300 μOhm cm. In another embodiment, the films deposited by the above methods and using the disclosed and claimed precursors have a resistivity below approximately 200 μOhm cm. In another embodiment, the films deposited by the above methods and using the disclosed and claimed precursors have a resistivity below approximately 100 μOhm cm.

### EXAMPLES

Reference will now be made to more specific embodiments of the present disclosure and experimental results that provide support for such embodiments. The examples are given below to more fully illustrate the disclosed and claimed subject matter and should not be construed as limiting the disclosed subject matter in any way.

It will be apparent to those skilled in the art that various modifications and variations can be made in the disclosed subject matter and specific examples provided herein without departing from the spirit or scope of the disclosed subject matter. Thus, it is intended that the disclosed subject matter, including the descriptions provided by the following examples, covers the modifications and variations of the disclosed subject matter that come within the scope of any claims and their equivalents.

### Materials and Methods

All reactions and manipulations described in the examples were conducted under a nitrogen atmosphere using an inert atmosphere glove box or standard Schlenk techniques. Anhydrous tetrahydrofuran (THF) and hexanes were used as received from Millipore-Sigma. Molybdenum acetate was used as received from Strem Chemicals. N, N′ -dialkylformamidinium sulphates were prepared according to the procedure reported by Hellmut, B. et al., Chemische Berichte, 98(8), 2754-61 (1965). The formamidinium sulphates were reacted with two equivalents of potassium hexamethyldisilazide to prepare the potassium formamidinate salts. Sec-butyliminopyrrolidine was prepared according to the procedure reported by Wasslen, Y. et al., Dalton Transaction, 39(38), 9046-9054 (2010) and reacted with potassium hexamethyldisilazide prior to use. Lithium N, N′ -diisopropylacetamidinate was prepared according to the procedure reported Coles, M. P. et al., Organometallics, 16(24), 5183-5194 (1997).

Single crystal X-ray diffraction studies were carried out on a Bruker D8 Venture diffractometer equipped with Mo K_{α} radiation (λ=0.71073 Å). Data were collected in a nitrogen gas stream at 100(2) K using ϕ and [symbol] scans.

Due to differences in instruments, samples, and sample preparation, peak values are reported with the modifier “about” in front of the peak values. This is common practice in the solid-state chemical arts because of the variation inherent in peak values. A typical precision of the 2θ x-axis value of a peak in a powder pattern is on the order of plus or minus 0.2° 2θ. Thus, a powder diffraction peak that appears at “about 9.2° 2θ,” means that the peak could be between 9.0° 2θ and 9.4° 2θ when measured on most X-ray diffractometers under most conditions.

### SPECIFIC EXAMPLES

### Example 1: Synthesis of Tetrakis(N,N′-dimethylformamidinate) Dimolybdenum (Table 7: Mo₂-(3A)₄ where M=Mo; aka Mo₂(Me-FMD)₄):

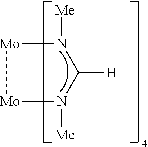

Molybdenum acetate (0.50 g, 1.17 mmol) was suspended in 15 mL of THF. Potassium N,N′-dimethylformamidinate (0.60 g, 5.44 mmol) dissolved in 5 mL of THF was added and the mixture stirred for 18 h. All volatile components were removed under reduced pressure to yield a solid. The solid was extracted with hexane (50 mL) followed by filtration to remove insoluble solids. The filtrate was reduced to dryness under reduced pressure to afford a yellow solid.

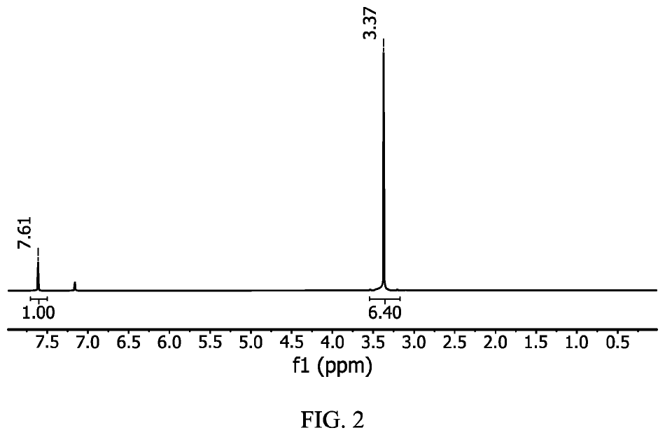

Analysis: Sublimation: 130° C. at 100 mTorr; ¹H NMR (C₆D₆, 25° C.): 3.37 (s, 24H), 7.61 (s, 4H). See FIG. 2.

### Example 2: Synthesis of Tetrakis(N,N′-dimethylformamidinate) Dimolybdenum (Table 7: Mo₂-(3A)₄ where M=Mo; aka Mo₂(Me-FMD)₄):

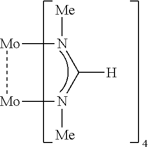

N,N′-dimethylformamidine (7.2 g, 100 mmol) was dissolved in 150 mL of THF. Sodium hydride (5 g, 210 mmol) was slowly added with vigorous stirring. The resulting suspension was stirred for 3 days at room temperature. The suspension was filtered and the filtrate evaporated to dryness under vacuum. The resulting off-white solid Sodium N,N′-dimethylformamidinate was used without additional purification. Sodium N,N′-dimethylformamidinate (9.4 g, 100 mmol) and Mo₂OAc₄ (10.7 g, 25 mmol) were combined in 400 mL of THF and stirred for 3 days at room temperature to form an orange solution with suspended solids. The THF was removed under vacuum and the resulting solid extracted with hexanes (3×250 mL) and filtered. The resulting hexanes solutions were combined and slowly evaporated to yield yellow-orange crystals.

Analysis: ¹H NMR (C₆D₆, 25° C.): 3.37 (s, 24H), 7.61 (s, 4H).

### Example 3: Crystal structure of Tetrakis(N,N′-dimethylformamidinate) Dimolybdenum (Table 7: Mo₂-(3A)₄ where M=Mo; aka Mo₂(Me-FMD)₄):

The crystals prepared in Example 2 were used to determine crystal structure of Mo₂-(3A)₄. The single crystal X-ray diffraction studies were carried out on a Bruker D8 Venture diffractometer equipped with Mo K_{α} radiation (X=0.71073 Å). A 0.20×0.20×0.25 mm piece of a yellow block was mounted on a Cryoloop with Paratone oil. Data were collected in a nitrogen gas stream at 100(2) K using ϕ and [symbol] scans. Crystal-to-detector distance was 50 mm and exposure time was 1 seconds per frame using a scan width of 0.7° . Data collection was 99.9% complete to 25.242° in θ. A total of 14961 reflections were and 1879 reflections were found to be symmetry independent, with a Rᵢₙₜ of 0.0266.

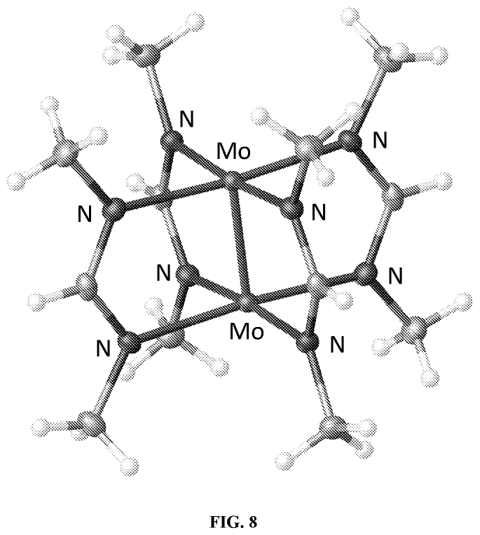

Indexing and unit cell refinement indicated a Primitive, Monoclinic lattice. The space group was found to be P2₁/n. The data were integrated using the Bruker SAINT software program and scaled using the SADABS software program. Solution by direct methods (SHELXT) produced a complete phasing model consistent with the proposed structure. All nonhydrogen atoms were refined anisotropically by full-matrix least-squares (SHELXL-2014). All carbon bonded hydrogen atoms were placed using a riding model. Their positions were constrained relative to their parent atom using the appropriate HFIX command in SHELXL-2014. See FIG. 8.

| Crystal system | Monoclinic | |
|---|---|---|
| Space group | P 1 21/n 1 | |
| Unit cell dimensions | a = 7.6761(3) Å | α = 90°. |
| | b = 7.5322(3) Å | β = 101.3520(10)°. |
| | c = 16.1584(6) Å | γ = 90°. |

| | |
|---|---|
| Volume | 915.97(6) Å₃ |
| Z | 2 |
| Density (calculated) | 1.727 Mg/m₃ |

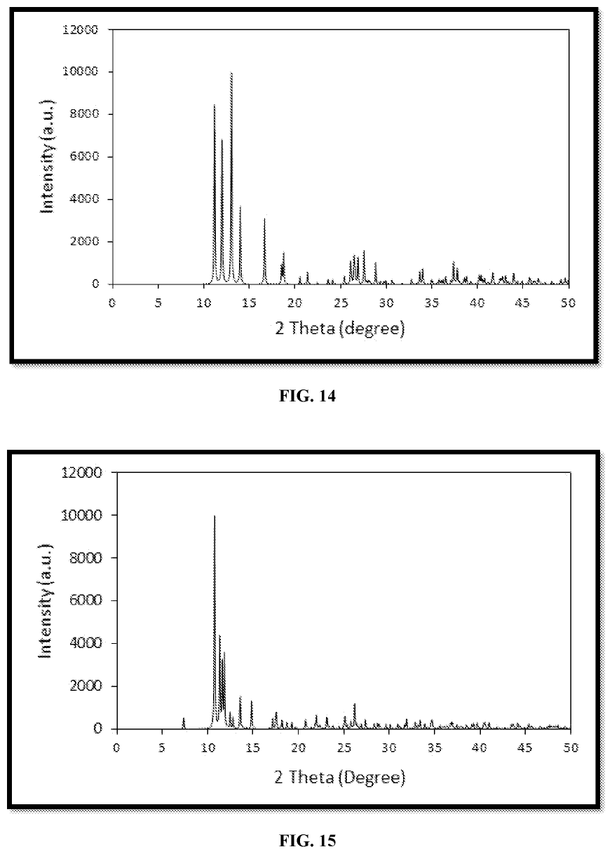

Selected peaks from simulated powder X-ray Diffraction (PXRD) spectrum using experimental unit cell parameters of Mo₂(Me-FMD)₄ and Cu Ka1 radiation source are as follow and are illustrated in FIG. 14.

| Peak | 2 Theta |
|---|---|
| No. | (degree ± 0.2) |
| 1 | 11.16 |
| 2 | 11.98 |
| 3 | 13.00 |
| 4 | 13.98 |
| 5 | 16.64 |
| 6 | 18.52 |
| 7 | 18.72 |
| 8 | 20.54 |
| 9 | 21.34 |
| 10 | 26.06 |
| 11 | 26.44 |
| 12 | 26.88 |
| 13 | 27.54 |
| 14 | 28.82 |
| 15 | 33.64 |
| 16 | 33.98 |
| 17 | 37.34 |
| 18 | 37.78 |

### Example 4: Synthesis of Tetrakis(N,N′-diethylformamidinate) Dimolybdenum (Table 7: Mo₂-(3B)₄ where M=Mo; aka Mo₂(Et-FMD)₄):

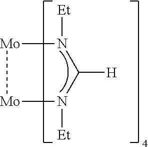

N,N′-diethylformamidinium sulfate (16.67 g, 73.7 mmol) was dissolved in 250 mL of THF. A solution of potassium hexamethyldisilazide (29.40 g, 147.4 mmol) in 125 mL of THF was added dropwise under vigorous stirring. The light-yellow slurry was stirred overnight. Mo₂OAc₄ (7.50 g, 17.5 mmol) was added as a solid and stirring continued over 4 days. All volatile components were removed under reduced pressure to yield a solid. The solid was extracted with hexane (3×50 mL). Each extraction was filtered to remove insoluble solids. The combined filtrates were reduced to dryness under reduced pressure to afford 7.50 g of a yellow solid.

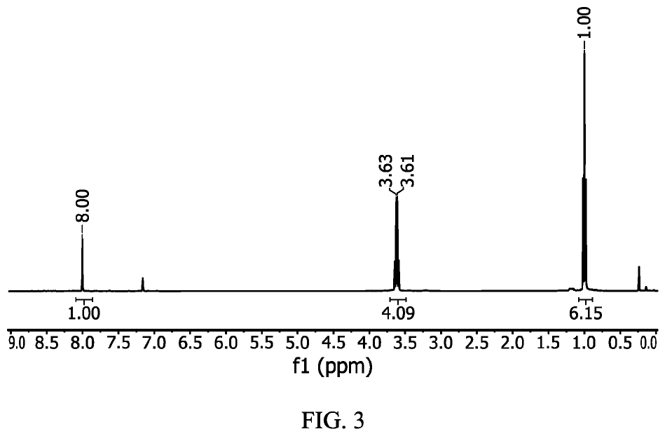

Analysis: Sublimation: 110-130° C., 100 mTorr, 6.4 g (60%); ¹H NMR (C₆D₆, 25° C.): 1.00 (t, 24H), 3.62 (q, 16H) 8.00 (s, 4H). See FIG. 3.

### Example 5: Synthesis of Tetrakis(N,N′-diethylformamidinate) Dimolybdenum (Table 7: Mo₂-(3B)₄ where M=Mo; aka Mo₂(Et-FMD)₄):

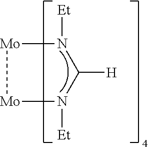

N,N′ -diethylformamidine (10 g, 100 mmol) was dissolved in 150 mL of THF. Sodium hydride (5 g, 210 mmol) was slowly added with vigorous stirring. The resulting suspension was stirred for 3 days at room temperature. The suspension was filtered, and the filtrate evaporated to dryness under vacuum. The resulting off-white solid Sodium N,N′-diethylformamidinate was used without additional purification. Sodium N,N′-diethylformamidinate (12.2 g, 100 mmol) and Mo₂OAc₄ (10.7 g, 25 mmol) were combined in 400 mL of THF and stirred for 3 days at room temperature to form an orange solution with suspended solids. The THF was removed under vacuum and the resulting solid extracted with hexanes (3×150 mL) and filtered. The resulting hexanes solutions were combined and slowly evaporated to yield orange crystals.

Analysis: ¹H NMR (C₆D₆, 25° C.): 1.00 (t, 24H), 3.62 (q, 16H) 8.00 (s, 4H).

### Example 6: Crystal Structure of Tetrakis(N,N′-diethylformamidinate) Dimolybdenum (Table 7: Mo₂-(3B)₄ where M=Mo; aka Mo₂(Et-FMD)₄)

The single crystal X-ray diffraction studies were carried out on a Bruker D8 Venture diffractometer equipped with Mo K_{α} radiation (λ=0.71073 Å). A 0.22×0.20×0.16 mm piece of a yellow block was mounted on a Cryoloop with Paratone oil. Data were collected in a nitrogen gas stream at 100(2) K using ϕ and [symbol] scans. Crystal-to-detector distance was 60 mm and exposure time was 3 seconds per frame using a scan width of 0.6° . Data collection was 99.9% complete to 25.242° in θ. A total of 48790 reflections were collected and 5259 reflections were found to be symmetry independent, with a Rᵢₙₜ of 0.0539.

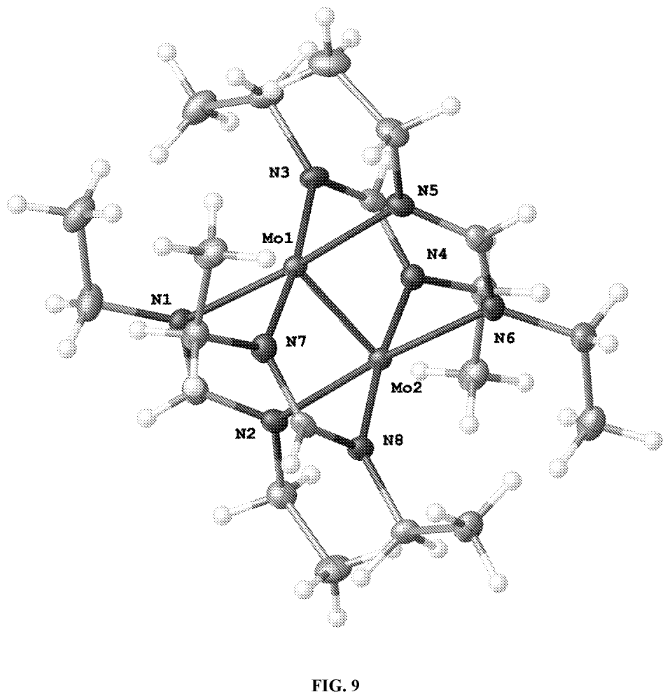

Indexing and unit cell refinement indicated a Primitive, Monoclinic lattice. The space group was found to be P2₁/n. The data were integrated using the Bruker SAINT software program and scaled using the SADABS software program. Solution by direct methods (SHELXT) produced a complete phasing model consistent with the proposed structure. All nonhydrogen atoms were refined anisotropically by full-matrix least-squares (SHELXL-2014). All carbon bonded hydrogen atoms were placed using a riding model. Their positions were constrained relative to their parent atom using the appropriate HFIX command in SHELXL-2014. See FIG. 9.

| Crystal system | Monoclinic | |
|---|---|---|
| Space group | P 1 21/n 1 | |
| Unit cell dimensions | a = 8.6456(5) Å | α = 90°. |
| | b = 19.4740(9) Å | β = 100.465(2)°. |
| | c = 15.4714(8) Å | γ = 90°. |

| | |
|---|---|
| Volume | 2561.5(2) Å₃ |
| Z | 4 |
| Density (calculated) | 1.526 Mg/m₃ |

Selected peaks from simulated powder X-ray Diffraction (PXRD) spectrum using experimental unit cell parameters of Mo₂(Et-FMD)₄ and Cu Ka1 radiation source are as follow and are illustrated in FIG. 15.

| Peak | 2 Theta |
|---|---|
| No. | (degree ± 0.2) |
| 1 | 7.36 |
| 2 | 10.78 |
| 3 | 11.36 |
| 4 | 11.62 |
| 5 | 11.86 |
| 6 | 13.6 |
| 7 | 14.84 |
| 8 | 17.18 |
| 9 | 17.56 |
| 10 | 20.78 |
| 11 | 22 |
| 12 | 23.14 |
| 13 | 25.14 |
| 14 | 26.2 |
| 15 | 31.9 |
| 16 | 32.86 |
| 17 | 33.4 |

### Example 7: Synthesis of Tetrakis(N-sec-butyliminopyrrolydinate) Dimolybdenum (Table 7: Mo₂-(3TT)₄ where M=Mo; aka Mo₂(sBu-IP)₄):

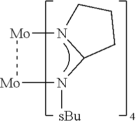

Following a similar procedure as described above for Example 1 (i.e., [Mo₂(Me-FMD)₄]) but using potassium N-sec-butyl-iminopyrrolidinate in place of potassium N,N′-dimethylformamidinate, a yellow solid (90%) was obtained.

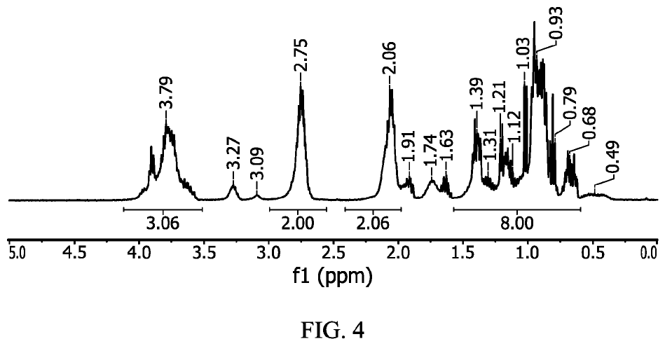

Analysis: Sublimation: 140° C. at 70 mTorr; ¹H NMR (C₆D₆, 25° C.): 0.60-1.70 (br, 24H), 2.00-2.20 (br, 8H), 2.60-3.00 (br, 8H), 3.50-4.10 (br, 12H). See FIG. 4.

### Example Deposition Methods

### Example 8: Thermal Chemical Vapor Deposition of Mo-Containing Films

The deposition experiment was conducted in 200 mm CN-1 shower head type deposition reactor. Mo₂(Et-FMD)₄, as produced in Example 4, was loaded into 200 sccm SS316 container, connected to deposition reactor delivery system and heated to 153° C. Sufficient vapor of the Mo₂(Et-FMD)₄ was delivered to deposition chamber by flow of 20 sccm of argon into container with Mo₂(Et-FMD)₄. Thermal cyclic CVD (CCVD) was demonstrated by 100 cycles of 5 sec of Mo₂(Et-FMD)₄/20 sec Ar purge over Si substrates heated to 250° C., 350 ° C. and 450° C. Thickness of molybdenum-containing films was measured by X-ray fluorescence (XRF). Almost no deposition was observed at 250° C. suggesting that the precursor is thermally stable up to at least this temperature and can be used for atomic layer deposition. At 350° C.˜40 Å and at 450° C.˜110 Å of molybdenum-containing films were deposited by thermal CVD. Film sheet resistance was measured by four-point probe electrode method. Film thickness and film resistance are summarized in Table 8. The experiment suggests that above 250° C. the disclosed and claimed precursors can be used for CVD or CCVD of molybdenum-containing films. Mo-containing film with low resistivity (<200 μOhm cm) was also demonstrated by thermal CVD at 450° C. It is also anticipated that films with even lower resistivity can be deposited by this process at higher deposition temperature.

TABLE 8
Thermal CVD of Molybdenum-Containing Films

| Wafer Temp | Film Thickness | | Film Resistivity |
|---|---|---|---|
| (° C.) | (Å) | Sheet Resistance | (μOhm cm) |
| 250 | 6.4 | No electrical contact | — |
| 350 | 39.3 | No electrical contact | — |
| 450 | 109.8 | 161.6 | 177.5 |

### Example 9: Ammonia Cyclic Chemical Vapor Deposition of Mo-Containing Films

The deposition experiment was conducted in 200 mm CN-1 shower head type deposition reactor. Mo₂(Et-FMD)₄, as produced in Example 4, was loaded into 200 sccm SS316 container, connected to deposition reactor delivery system and heated to 153° C. Sufficient vapor of the Mo₂(Et-FMD)₄ was delivered to deposition chamber by flow of 20 sccm of argon into container with Mo₂(Et-FMD)₄. Ammonia cyclic CVD (CCVD) was demonstrated by 100 cycles of pulsed process using pulses of molybdenum precursor and ammonia co-reagent with argon purge between precursor and co-reagent pulses: 10 or 20 sec of Mo₂(Et-FMD)₄/30 sec Ar purge/5 sec of NH₃/30 sec of Ar purge. The films were deposited on Si substrates at 300° C. and 350° C. Thickness of molybdenum-containing films was measured by X-ray fluorescence (XRF). Film sheet resistance was measured by four-point probe electrode method. Film thickness and film resistance are summarized in Table 9. The experiment shows that the addition of NH₃ pulse increases deposition rate of Mo-containing film. Deposition of molybdenum-containing film with low resistivity (<200 μOhm cm) was demonstrated by ammonia cyclic thermal CVD without plasma assistance. The ability to deposit low resistivity molybdenum-containing film by a halogen-free thermal deposition process is one of the advantages of the disclosed and claimed precursors.

TABLE 9
Ammonia Cyclic CVD of Molybdenum-Containing Films

| Wafer | Pulse Sequence: | Film | | Film |
|---|---|---|---|---|
| Temp | Mo₂(Et-FMD)₄/ | Thickness | Sheet | Resistivity |
| (° C.) | Ar/NH₃/Ar (sec) | (Å) | Resistance | (μOhm cm) |
| 300 | 10/30/5/30 | 18.0 | No electrical | — |
| | | | contact | |
| 300 | 20/30/5/30 | 30.2 | No electrical | — |
| | | | contact | |
| 350 | 10/30/5/30 | 229.0 | 151.41 | 346.7 |
| 350 | 20/30/5/30 | 123.9 | 153.10 | 189.7 |

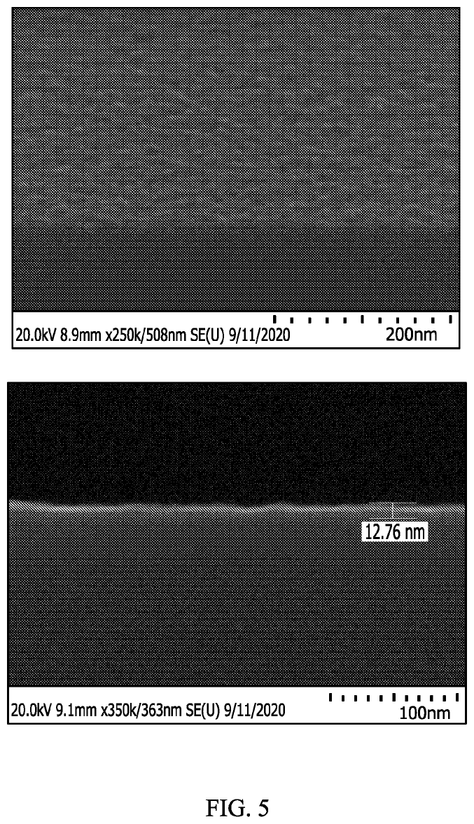

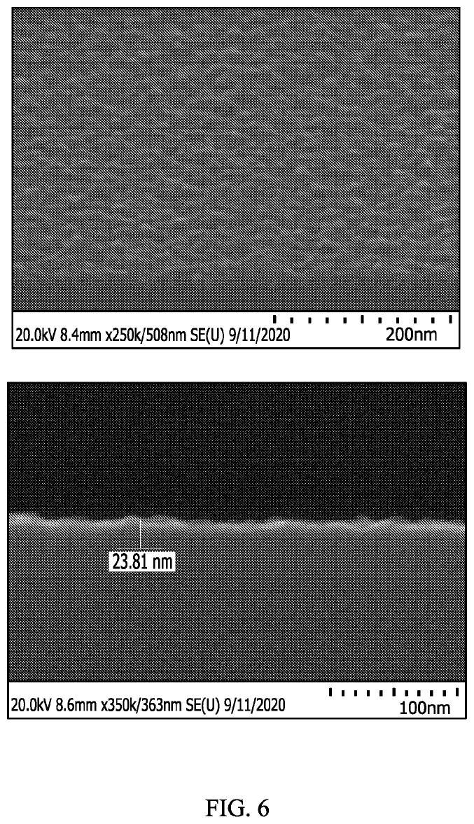

FIGS. 5 and 6 show SEM of Mo-containing films deposited by ammonia cyclic CVD process.

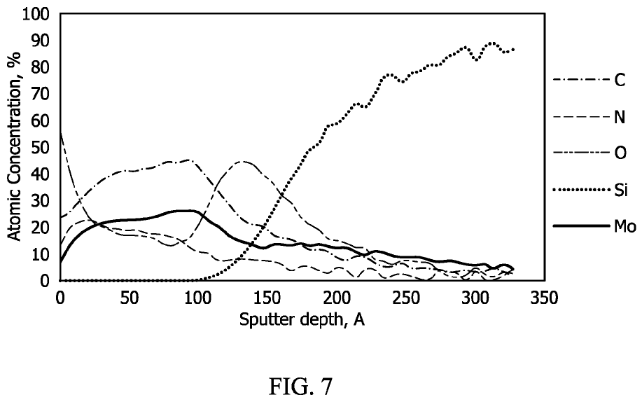

FIG. 7 shows Auger depth profile of Mo-containing films deposited by ammonia cyclic CVD process and demonstrates incorporation of nitrogen into the film by ammonia cyclic CVD.

### Example 10: Hydrogen Plasma Cyclic Chemical Vapor Deposition of Mo-Containing Films

The deposition experiment was conducted in 200 mm CN-1 shower head type deposition reactor. Mo₂(Et-FMD)₄, as produced in Example 4, was loaded into 200 sccm SS316 container, connected to deposition reactor delivery system and heated to 153° C. Sufficient vapor of the Mo₂(Et-FMD)₄ was delivered to deposition chamber by flow of 20 sccm of argon into container with Mo₂(Et-FMD)₄. Hydrogen plasma cyclic CVD (CCVD) was demonstrated by 100 cycles of pulsed process using pulses of molybdenum precursor and hydrogen plasma co-reagent with argon purge between precursor and co-reagent pulses: 10 sec of Mo₂(Et-FMD)₄/30 sec Ar purge/5 sec of Hydrogen plasma with a 175-watt RF power/30 sec of Ar purge. The films were deposited on Si and TiN substrates at 350° C. Thickness of molybdenum-containing films was measured by X-ray fluorescence (XRF). Film sheet resistance was measured by four-point probe electrode method. Film thickness and film resistance are summarized in Table 10. The experiment shows that addition of hydrogen-plasma step further reduces film resistivity to as low as 137 μOhm cm.

TABLE 10
Hydrogen Cyclic CVD of Molybdenum-Containing Films

| | Film Thickness | | Film resistivity |
|---|---|---|---|
| Substrate | (Å) | Sheet Resistance | (μOhm cm) |
| TiN | 118.9 | Not measured | — |
| TiN | 126.8 | Not measured | — |
| Si | 98.9 | 153.64 | 151.9 |
| Si | 89.5 | 152.94 | 136.8 |

Low resistivity molybdenum containing thin films were deposited in a method typical for thermal atomic layer deposition. The method used ammonia gas as a co-reagent and argon as a purge gas. Each pulse of molybdenum precursor and ammonia were separated by purge pulses. The method was compared to a method where hydrogen plasma was used as the co-reagent. The thermal ammonia method afforded molybdenum containing films with resistivity values <300 μΩ·cm. The hydrogen plasma method afforded molybdenum containing films with resistivity values <200 μΩ·cm.

### Example 11: ALD of Mo-Containing Films on Silicon Oxide Patterned Wafers

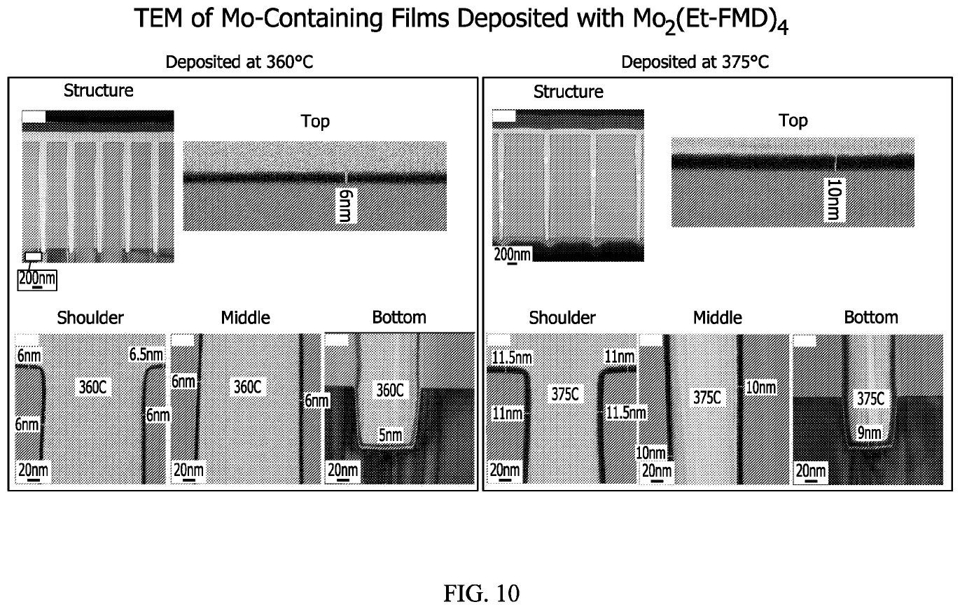

The deposition experiment was conducted in 200 mm CN-1 shower head type deposition reactor. Mo₂(Et-FMD)₄, as produced in Example 4, was loaded into 200 sccm SS316 container, connected to deposition reactor delivery system and heated to 160° C. Sufficient vapor of the Mo₂(Et-FMD)₄ was delivered to the deposition chamber by flow of 20 sccm of argon into container with Mo₂(Et-FMD)₄. ALD of Mo-containing films was demonstrated by 100 cycles of pulsed process using pulses of molybdenum precursor and ammonia co-reagent with argon purge between precursor and co-reagent pulses: 10 sec of Mo₂(Et-FMD)₄/30 sec Ar purge/5 sec of NH3/30 sec of Ar purge. Chamber pressure was 20 torr. Film thickness on top, middle and bottom of patterned substrate were measured by TEM. Aspect ratio of the structure (A/R) was calculated by dividing the total structure depth (26000 Å) by structure width in the middle of the structure (1818 Å), as shown in FIG. 10. Middle A/R was calculated by dividing the depth at the middle (13000 Å) by the width at the middle of the structure (1818 Å). Bottom A/R was calculated by dividing the depth at the bottom (24725 Å) by the width at the bottom of the structure (1090 Å).

TABLE 11
Step coverage of MoCN films deposited by Mo₂(Et-FMD)₄

| | | | | | Step Coverage, % | | Deposition |
|---|---|---|---|---|---|---|---|
| Wafer | # of | Film Thickness, Å | | | Middle, | Bottom, | rate, top: |
| T, ° C. | cycles | Top | Middle | Bottom | A/R = 7.2 | A/R = 22.7 | Å/cycle |
| 360 | 100 | 61 | 60 | 50 | 98 | 82 | 0.61 |
| 375 | 150 | 115 | 100 | 90 | 87 | 78 | 0.77 |

This example shows that the precursors of this invention enable conformal deposition of low resistivity MoCN films on high aspect ratio structures.

### Example 12: Deposition of Thin Continuous MoCN Film on Silicon Oxide

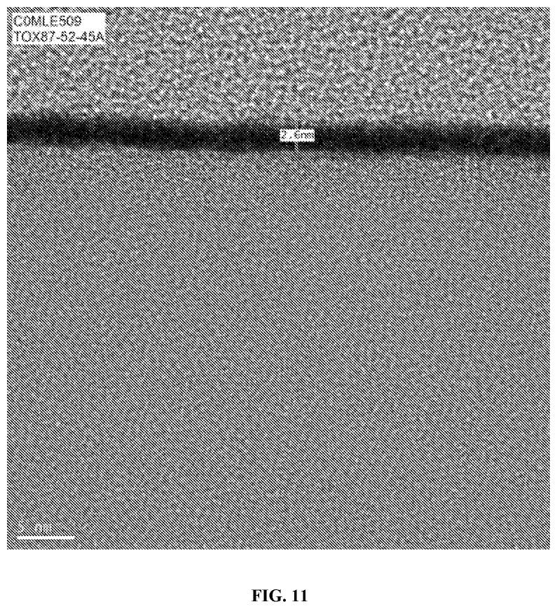

The deposition experiment was conducted in 200 mm CN-1 shower head type deposition reactor. Mo₂(Me-FMD)₄, as produced in Example 2, was loaded into 200 sccm SS316 container, connected to deposition reactor delivery system and heated to 160° C. Sufficient vapor of the Mo₂(Me-FMD)₄ was delivered to deposition chamber by flow of 20 sccm of argon into container with Mo₂(Me-FMD)₄. Continuous 2.6 nm MoCN film was deposited by ammonia cyclic CVD (CCVD) by 30 cycles of pulsed process using pulses of Mo₂(Me-FMD)₄ and ammonia co-reagent with argon purge between precursor and co-reagent pulses: 10 sec of Mo₂(Me-FMD)₄/30 sec Ar purge/10 sec of NH₃/10 sec of Ar purge. The film was deposited on thermal silicon oxide at 400° C. Thickness of molybdenum-containing films was measured by TEM and is shown on FIG. 11. Sheet resistance of this film was measured by four points electrode method, 7850 Ohm sq, which correspond to thin film resistivity of 2041 μOhm cm. The example demonstrates that precursors of this invention enable deposition of thin continuous conductive MoCN films on silicon oxide substrates.

### Example 13: Characterization of Mo-Containing Film

The deposition experiment was conducted in 200 mm CN-1 shower head type deposition reactor. Mo₂(Me-FMD)₄, as produced in Example 2, was loaded into 200 sccm SS316 container, connected to deposition reactor delivery system and heated to 160° C. Sufficient vapor of the Mo₂(Me-FMD)₄ was delivered to deposition chamber by flow of 20 sccm of argon into a container with Mo₂(Me-FMD)₄. MoCN films were deposited by ammonia cyclic CVD (CCVD) using pulses of Mo₂ (Me-FMD)₄ and ammonia co-reagent with argon purge between precursor and co-reagent pulses: 10 sec of Mo₂(Me-FMD)₄/30 sec Ar purge/10 sec of NH₃/10 sec of Ar purge. The films were deposited on thermal silicon oxide at 350 and 400° C. Thickness of molybdenum-containing films was measured by XRR and film composition was measured by XPS, Table 12. The example demonstrates deposition of low resistivity of MoCxNy films on silicon oxide substrate using precursors of the invention, where x is ranging from approximately 0.5 to 1 and N is below 0.5.

TABLE 12
Resistivity and film composition deposited with Mo₂(Me-FMD)₄

| Wafer | # of | Thickness, | Sheet R, | Resistivity, | Mo | C, | N, | Film |
|---|---|---|---|---|---|---|---|---|
| T, ° C. | cycles | A | Ohm sq | μOhm cm | at % | at % | at % | Composition |
| 350 | 210 | 90 | 688 | 619 | 40.7 | 33.9 | 16.0 | MoC_{0.83}N_{0.39} |
| 400 | 170 | 86 | 332 | 286 | 43.7 | 25.2 | 17.0 | MoC_{0.58}N_{0.40} |

### Example 14: Plasma Enhanced Deposition of Mo-Containing Film on Silicon Oxide

The deposition experiment was conducted in 200 mm CN-1 shower head type deposition reactor. Mo₂(Me-FMD)₄, as produced in Example 2, was loaded into 200 sccm SS316 container, connected to deposition reactor delivery system and heated to 160° C. Sufficient vapor of the Mo₂(Et-FMD)₄ was delivered to deposition chamber by flow of 20 sccm of argon into container with Mo₂(Me-FMD)₄. MoCN films were deposited by cyclic CVD (CCVD) using pulses of Mo₂(Me-FMD)₄ and hydrogen, nitrogen or ammonia co-reagent with argon purge between precursor and co-reagent pulses: 10 sec of Mo₂(Et-FMD)₄/30 sec Ar purge/10 sec of NH₃/10 sec of Ar purge. During co-reagent pulse RF plasma was applied. The films were deposited on thermal silicon oxide at 250° C. and 350° C. Thickness of molybdenum-containing films was measured by XRF and film sheet resistance was measured by four-point probe method Table 13. The example demonstrates deposition of low resistivity MoCN films on silicon oxide substrate using precursors of the invention, where the thickness of MoCN film is <30 Å and resistivity is as low as approximately 300 μOhm cm.

TABLE 13
Film thickness and resistivity of films
deposited by PEALD with Mo₂(Me-FMD)₄

| Wafer | | Plasma | Film Thk. on | Film Resistivity, |
|---|---|---|---|---|
| T, ° C. | Plasma | Power, W | SiO₂, Å | μOhm cm |
| 250 | H₂ | 125 | 26.0 | 417 |
| 250 | N₂ | 125 | 18.1 | 6390 |
| 250 | NH₃ | 200 | 18.3 | 9272 |
| 350 | H₂ | 125 | 26.7 | 316 |
| 350 | N₂ | 125 | 20.4 | 6609 |
| 350 | NH₃ | 200 | 24.9 | 3037 |

### Example 15: Deposition of Mo Metal Film by MoO₂Cl₂/H₂ process on silicon oxide Substrate with and without Seed Layer Deposited by Mo₂(Me-FMD)₄

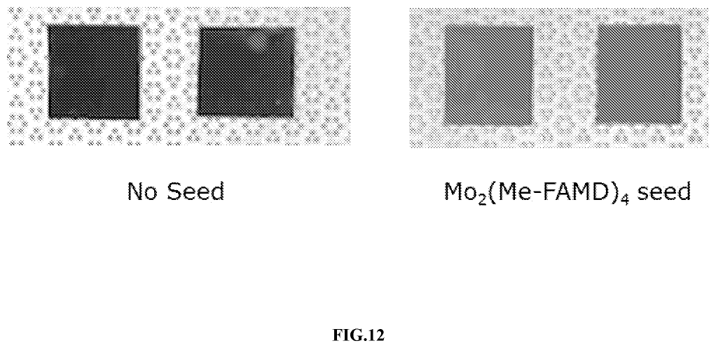

In this experiment Mo metal film was attempted to deposit directly on silicon oxide substrate at 500° C. wafer temperature by 150 cycles of the following process: 2 sec of MoO₂Cl₂/6 sec of Ar purge/10 sec of H₂/5 sec of Ar purge (chamber pressure was 30 torr). MoO₂Cl₂ was purchased from Sigma Aldrich and delivered from a 316SS container heated to 60° C. No Mo deposition was observed on silicon oxide wafer as shown in FIG. 12 In another experiment Mo metal film was deposited using 150 cycles of the same MoO₂Cl₂/H₂ process but on a seed layer deposited by 30 cycles of the following process: 10 sec of Mo₂(Me-FMD)₄/30 sec Ar purge/10 sec of NH₃/10 sec of Ar purge (chamber pressure was 1 torr, wafer temperature was 400° C.). Continuous Mo film was deposited when Mo₂(Me-FMD)₄ is used as shown in FIG. 12.

### Example 16: Deposition of Mo-Metal Film by MoO₂Cl₂/H₂ Process on a Patterned Silicon Oxide Substrate Using Seed Layer Deposited by Mo₂(Me-FMD)₄

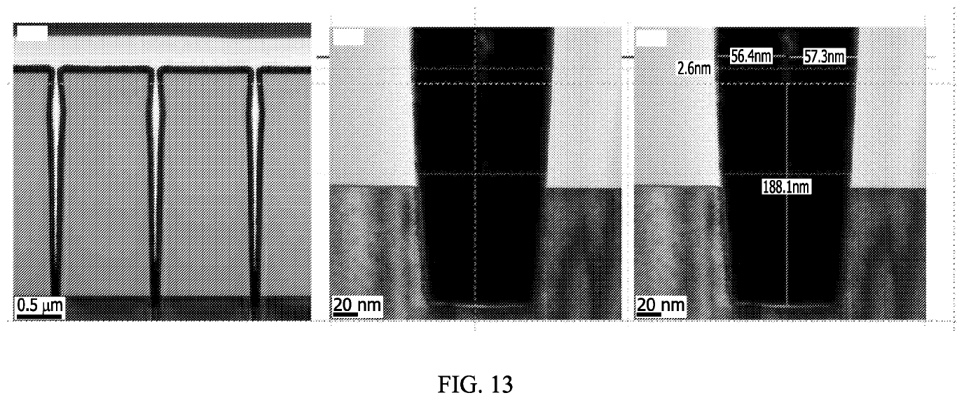

In this experiment Mo metal film was deposited on a patterned silicon oxide substrate. In the first step of the process the seed layer was deposited at 400° C. wafer temperature by 30 cycles of the following sequence: 10 sec of Mo₂(Me-FMD)₄/30 sec Ar purge/10 sec of NH₃/10 sec of Ar purge (chamber pressure was 1 torr). Following this step bulk Mo metal film was deposited at 500° C. by 1100 cycles of the following sequence: 2 sec of MoO₂Cl₂/6 sec of Ar purge/10 sec of H₂/5 sec of Ar purge (chamber pressure was 30 torr). MoO₂Cl₂ was purchased from Sigma Aldrich and delivered from a 316SS container heated to 60° C. FIG. 13 shows conformal fill of patterned silicon oxide substrate by the process of the invention.

It is anticipated that the invented method could be used in conjunction with deposition tools commonly found at semiconductor manufacturing sites to produce molybdenum-containing layers for logic applications and other potential functions.

### SUMMARY OF EXAMPLES

This class of compound provides halide-free and oxygen-free precursors for applications where such contaminates are detrimental. Precursor properties, such as thermal stability, volatility, and composition, are optimal when the amidinate ligand is a formamidinate. Specifically, when the nitrogen alkyl substituents are small (C₁-C₅) and the exocyclic substituent of the cyclic carbon of the amidine ligand is a hydrogen atom. Another suitable amidine ligand is the iminopyrrolidinate. This amidine is monocyclic and asymmetric. Metal and metal-containing thin films can be produced by thermal or plasma Atomic Layer Deposition and Chemical Vapor Deposition. The method produces thin films with improved properties compared to known methods which could be attributed to the low oxidation state of the paddlewheel precursor.

A homoleptic molybdenum (II) amidinate or guanidinate paddlewheel compound is obtained by appropriate selection of the ligand. Small amidines (such as formamidines), small guanidines, or sterically unencumbered mono-cyclic and bi-cyclic amidines and guanidines (e.g., iminopyrrolidines) form homoleptic molybdenum (II) paddlewheel compounds. Whereas the larger amidine, N,N-di-isopropyl-acetamidine, forms a heteroleptic molybdenum (II) amidinate paddlewheel compound. Avoiding oxygen in the ligand composition and in the metal coordination sphere eliminates the possibility of oxygen contamination during the thin film deposition process.

The described method provides molybdenum films by low temperature, thermal Atomic Layer Deposition. A low temperature, thermal process provides better integration into existing semiconductor manufacturing methods, better materials compatibility than higher temperature processes, and enables lower thermal budget. The ability to produce molybdenum films using ALD provides the advantages inherent to that film growth method, including high uniformity of thickness, ability to coat high aspect ratio features, and precise control of film thickness for very thin layers. Additionally, due to the design of the precursor, thin film contaminates such as oxygen and halides are avoided. The rigid paddlewheel structure improves the shelf-life of the precursor at the required container temperature. The low oxidation state of the molybdenum atoms deposits electron-rich thin films with desirable electrical properties. In addition, the deposition rate is more than double compared to MoBure.

The foregoing description is intended primarily for purposes of illustration. Although the disclosed and claimed subject matter has been shown and described with respect to an exemplary embodiment thereof, it should be understood by those skilled in the art that the foregoing and various other changes, omissions, and additions in the form and detail thereof may be made therein without departing from the spirit and scope of the disclosed and claimed subject matter.

1. A precursor of formula I: 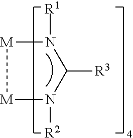
or formula II: 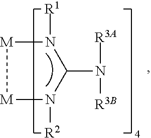
wherein M is one of chromium, molybdenum and tungsten; and
(ii) R¹, R², R³, R^{3A} and R^{3B} are each independently selected from H, D, an unsubstituted linear C₁-C₆alkyl group, a linear C₁-C₆ alkyl group substituted with a halogen, a linear C₁-C₆ alkyl group substituted with an amino group, an unsubstituted branched C₃-C₆ alkyl group, a branched C₃-C₆ alkyl group substituted with a halogen, a branched C₃-C₆ alkyl group substituted with an amino group, an unsubstituted amine, an substituted amine, —Si(CH₃)₃, a C₃-C₈ unsubstituted cyclic alkyl group, a C₃-C₈ cyclic alkyl group substituted with a halogen, a C₃-C₈ cyclic alkyl group substituted with an amino group, a C₃-C₈ unsubstituted aromatic group, a C₃-C₈ aromatic group substituted with a halogen and a C₃-C₈ aromatic group substituted with an amino group.

2. The precursor of claim 1, wherein M is chromium.

3. The precursor of claim 1, wherein M is molybdenum.

4. The precursor of claim 1, wherein M is tungsten.

5. The precursor of claim 1, wherein all four Amidinatc ligands have the same chemical structure (ii) R¹, R², R³, R^{3A} and R^{3B} are each independently selected from H and an unsubstituted linear C₁-C₆ alkyl group.

6. The precursor of claim 1, wherein two or more of the Amidinatc ligands have the same chemical structure each of R¹ and R² is a methyl group.

7. The precursor of claim 1, wherein all four Amidinatc ligands have a different chemical structure each of R¹ and R² is an ethyl group.

8. (canceled)

9. (canceled)

10. The precursor of claim 1, wherein one of R¹ and R² is a methyl group and the other of R¹ and R² is an ethyl group.

11. The precursor of claim 1, wherein R³ is H.

12. The precursor of claim 1, each of R^{3A} and R^{3B} is a methyl group.

13. The precursor of claim 1, each of R^{3A} and R^{3B} is an ethyl group.

14-18. (canceled)

19. The precursor of claim 1 having the formula:

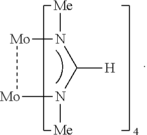

20. The precursor of claim 19 characterized by an x-ray powder diffraction pattern substantially in accordance with that shown in FIG. 14.

21. The precursor of claim 19 characterized by an x-ray powder diffraction pattern comprising four or more 2θ values selected from the group consisting of:

| Peak | 2 Theta |
|---|---|
| No. | (degree ± 0.2) |
| 1 | 11.16 ± 0.2 |
| 2 | 11.98 ± 0.2 |
| 3 | 13.00 ± 0.2 |
| 4 | 13.98 ± 0.2 |
| 5 | 16.64 ± 0.2 |
| 6 | 18.52 ± 0.2 |
| 7 | 18.72 ± 0.2 |
| 8 | 20.54 ± 0.2 |
| 9 | 21.34 ± 0.2 |
| 10 | 26.06 ± 0.2 |
| 11 | 26.44 ± 0.2 |
| 12 | 26.88 ± 0.2 |
| 13 | 27.54 ± 0.2 |
| 14 | 28.82 ± 0.2 |
| 15 | 33.64 ± 0.2 |
| 16 | 33.98 ± 0.2 |
| 17 | 37.34 ± 0.2 |
| 18 | 37.78 ± 0.2 |

22. The precursor of claim 19 characterized by an x-ray powder diffraction pattern having characteristic peaks at 11.16±0.2, 11.98±0.2, 13.00±0.2, 13.98±0.2 and 16.64±0.2 degrees 20.

23. The precursor of claim 1 having the formula:

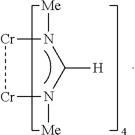

24. The precursor of claim 1 having the formula:

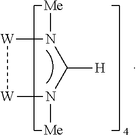

25. The precursor of claim 1 having the formula:

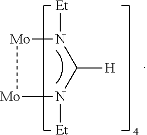

26. The precursor of claim 25 characterized by an x-ray powder diffraction pattern substantially in accordance with that shown in FIG. 15.

27. The precursor of claim 25 characterized by an x-ray powder diffraction pattern comprising four or more 20 values selected from the group consisting of:

| Peak | 2 Theta |
|---|---|
| No. | (degree ± 0.2) |
| 1 | 7.36 ± 0.2 |
| 2 | 10.78 ± 0.2 |
| 3 | 11.36 ± 0.2 |
| 4 | 11.62 ± 0.2 |
| 5 | 11.86 ± 0.2 |
| 6 | 13.6 ± 0.2 |
| 7 | 14.84 ± 0.2 |
| 8 | 17.18 ± 0.2 |
| 9 | 17.56 ± 0.2 |
| 10 | 20.78 ± 0.2 |
| 11 | 22 ± 0.2 |
| 12 | 23.14 ± 0.2 |
| 13 | 25.14 ± 0.2 |
| 14 | 26.2 ± 0.2 |
| 15 | 31.9 ± 0.2 |
| 16 | 32.86 ± 0.2 |
| 17 | 33.4 ± 0.2 |

28. The precursor of claim 25 characterized by an x-ray powder diffraction pattern having characteristic peaks at 10.78±0.2, 11.36±0.2, 11.62±0.2, 11.86±0.2 and 13.6±0.2 degrees 20.

29. The precursor of claim 1 having the formula:

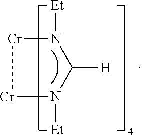

30. The precursor of claim 1 having the formula:

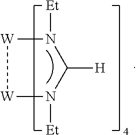

31-37. (canceled)

38. The precursor of claim 1 having the formula:

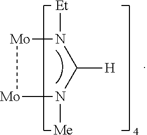

39. The precursor of claim 1 having the formula:

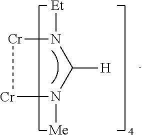

40. The precursor of claim 1 having the formula:

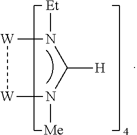

41. The precursor of claim 1 having the formula:

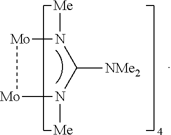

42. The precursor of claim 1 having the formula:

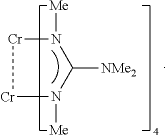

43. The precursor of claim 1 having the formula:

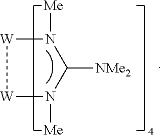

44-52. (canceled)

53. A method for forming a transition metal-containing film on at least one surface of a substrate comprising:
a. providing the at least one surface of the substrate in a reaction vessel;
b. forming a transition metal-containing film on the at least one surface by a thermal deposition process chosen from a chemical vapor deposition (CVD) process and an atomic layer deposition (ALD) process using one or more precursors of claim 1 as a metal source compound for the deposition process.

54-85. (canceled)
